(19) United States
(12) Patent Application Publication
(10) Pub. No.: US 2001/0012845 A1
(43) Pub. Date: Aug. 9, 2001

(54) COMBINATION PRODUCT AND METHODS OF USE

(76) Inventors: CHI CHUNG CHAN, KIRKLAND (CA); MARC LABELLE, LLE PERROT (CA); KATHLEEN METTERS, MONTREAL (CA)

Correspondence Address: RICHARD C BILLUPS, PATENT DEPARTMENT, MERCK CO INC, P O BOX 2000, RAHWAY, NJ, 070650907

(21) Appl. No.: 09/425,376

(22) Filed: Oct. 22, 1999

Related U.S. Application Data
(60) Provisional application No. 60/105,837, filed on Oct. 23, 1998.

(51) Int. Cl. A61K 31/44; A61K 31/47

(52) U.S. Cl. 514/309

(57) ABSTRACT

A pharmaceutical composition is disclosed which is comprised of an E-type prostaglandin ligand and a COX-2 selective inhibiting compound, in combination with a pharmaceutically acceptable carrier. Methods of treatment are also disclosed wherein an E-type prostaglandin ligand and a COX-2 selective inhibiting compound are adminstered in an amount that is effective to treat or prevent an E-type prostaglandin and/or COX-2 mediated disease or condition.

### BACKGROUND OF THE INVENTION

This invention relates to combinations of compounds and methods for treating or preventing E-type prostaglandin and COX-2 mediated diseases, and pharmaceutical compositions that contain such compounds. More particularly, the combinations of compounds are antagonists of the pain and inflammatory effects of E-type prostaglandins and COX-2.

Two review articles describe the characterization and therapeutic relevance of the prostanoid receptors as well as the most commonly used selective agonists and antagonists: Eicosanoids: From Biotechnology to Therapeutic Applications, Folco, Samuelsson, Maclouf, and Velo eds, Plenum Press, New York, 1996, chap. 14,137-154 and Journal of Lipid Mediators and Cell Signalling, 1996, 14, 83-87. An article from The British Journal of Pharmacology (1994, 112, 735-740) suggests that Prostaglandin E₂ (PGE₂) exerts allodynia through the EP₁ receptor subtype and hyperalgesia through EP2 and EP₃ receptors in the mouse spinal cord.

As prostaglandins have both physiological and pathological roles, the constitutive enzyme, COX-1, is responsible, in large part, for endogenous basal release of prostaglandins and hence is important in their physiological functions such as the maintenance of gastrointestinal integrity and renal blood flow. In contrast, the inducible form, COX-2, is mainly responsible for the pathological effects of prostaglandins where rapid induction of the enzyme would occur in response to such agents as inflammatory agents, hormones, growth factors, and cytokines.

Selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects.

The potential utilities of selective cyclooxygenase-2 inhibitors are discussed in John Vane, “Towards a better aspirin” in Nature, Vol. 367, pp. 215-216, 1994; in Drug News and Perspectives, Vol. 7, pp. 501-512, 1994; and David B. Reitz and Karen Seibert, “Selective Cyclooxygenase Inhibitors” in Annual Reports in Medicinal Chemistry.

These compounds in combination have a diminished ability to induce some of the mechanism-based side effects of NSAIDs which are indiscriminate cyclooxygenase inhibitors. In particular, the combination has a reduced potential for gastrointestinal toxicity, a reduced potential for renal side effects, a reduced effect on bleeding times and a lessened ability to induce asthma attacks in aspirin-sensitive asthmatic subjects. In addition, the combination of compounds is unexpectedly potent in its analgesic potency.

PCT application nos WO 96/06822 (Mar. 7, 1996), WO 96111902 (Apr. 25, 1996), WO 97/00863 (Jan. 9, 1997), WO 97/00864 (Jan. 9, 1997), WO 96/03380 (Feb. 8, 1996), and EP 752421-A1 (Jan. 08, 1997) disclose compounds represented by Formula I as being useful in the treatment of prostaglandin mediated diseases.

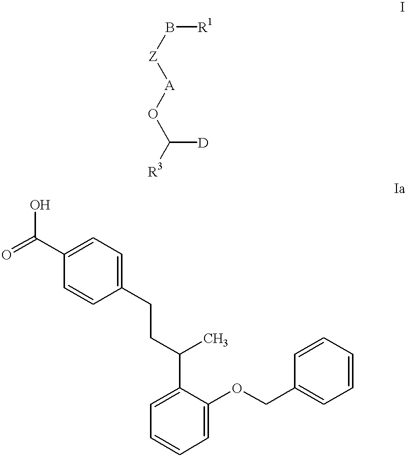

wherein:

A is a phenyl, naphthyl, 5- or 6-membered heteroaryl group;

B is phenyl, 5- or 6-membered heteroaryl or a further defined keto-dihydro ring;

D is phenyl, 5- or 6-membered heteroaryl;

R¹ is COOH, carboxyalkyl, tetrazolyl(alkyl);

R³ is H or alkyl, and

Z is an alkylene bridge containing 0-1 nitrogen atom or a further defined unsaturated bridge.

Compound Ia is one of the compounds specifically claimed.

Additionally, U.S. application Ser. No. 60/077,990 filed on Mar. 13, 1998 and provisional patent application nos. 60/103564 (Merck Case No. 20255PV) and 60/103,371 (Merck Case No. 20085PV) filed on Oct. 7, 1998 address compounds which are ligands of E-type prostaglandins, and hence useful in the invention described herein.

Numerous patents and patent applications disclose compounds which are COX-2 selective inhibitors. Examples of COX-2 selective compounds are such as those described in the following patents and published applications: WO 96/25405, U.S.Pat. No. 5,633,272, WO 97/38986, U. S. Pat. No. 5,466,823, WO 98/03484, WO 97/14691 and WO 95/00501. Numerous other patents and published applications are available which disclose compounds as having COX-2 selectivity. However, the combination of an E-type prostaglandin ligand and a COX-2 selective inhibiting comound and use of these compounds in combination are new.

### SUMMARY OF THE INVENTION

In one aspect, the invention relates to a composition containing an E-type prostaglandin ligand and a COX-2 selective inhibiting compound, in combination with a pharmaceutically acceptable carrier.

The invention further relates to a method of treating or preventing an E-type prostaglandin and/or a COX-2 mediated disease or condition, which is comprised of admininstering to a mammalian patient in need thereof, an E-type prostaglandin ligand and a COX-2 selective inhibiting compound, in an amount which is effective to treat or prevent said disease or condition.

### DETAILED DESCRIPTION OF THE INVENTION

In one aspect, the invention relates to a composition containing an E-type prostaglandin ligand and a COX-2 selective inhibiting compound.

Examples of E-type prostaglandin ligands include compounds found in the published applications noted above, as well as in U.S. application Ser. No. 60/103564 (Merck Case No. 20255PV) filed on Oct. 7, 1998, addressing compounds represented by formula II:

Ar¹—W—Ar²—X—Q II

as well as pharmaceutically acceptable salts and hydrates thereof, wherein:

Ar¹ is an aryl or heteroaryl group, optionally substituted with R¹ or R³;

R¹ is Yₘ—R²Yₘ—Ar, halogen, N(R⁵)₂, CN, NO₂, C(R⁶)₃, CON(R⁵)₂, S(O)ₙR⁷ or OH;

Y represents a linker between R² or Ar³ and Ar¹ containing 0-4 carbon atoms and not more than one heteroatom selected from O, N and S, said linker optionally containing CO, S(O)n, —C═C— or an acetylenic group, and said linker being optionally substituted by R²;

m is 0 or 1;

n is 0, 1 or 2;

R² represents H, F, CHF₂, CF₃, lower alkyl or hydroxy C₁-C₆ alkyl, or two R² groups may be joined together and represent a carbocyclic ring of up to six members, said ring containing not more than one heteroatom selected from O, N and S;

Ar³ represents an aryl or heteroaryl group, optionally substituted with R³;

R³ is R⁴, halogen, halo C₁₋₆alkyl, N(R⁵)₂, CN, NO₂, C(R⁶)₃, CON(R⁵)₂, OR⁴, SR⁴ or S(O)ₙR⁷;

R⁴ is H, lower alkyl, lower alkenyl, lower alkynyl, CHF₂ or CF₃;

R⁵ is R⁴, Ph or Bn, or two R⁵ groups in combination with the atom to which they are attached represent a ring of up to 6 members containing carbon atoms and up to 2 heteroatoms selected from O, N and S;

R⁶ is H, F, CF₃ or lower alkyl, or two R⁶ groups may be taken together and represent a ring of up to 6 members containing carbon atoms and 0-2 heteroatoms selected from O, N and S;

R⁷ is lower alkyl, lower alkenyl, lower alkynyl, CHF₂, CF₃, N(R⁵)₂, Ph(R⁸)₂ or CH₂Ph(R⁸)₂;

R⁸ is R⁴, OR⁴, SR⁴ or halogen

W represents a 3-6 membered linking group containing 0 to 2 heteroatoms selected from O, N and S, said linking group optionally containing CO, S(O)ₙ, C═C or an acetylenic group, and optionally being substituted with R⁹;

R⁹ is R², lower alkenyl, lower alkynyl, OR⁴ or SR⁴;

Ar² represents an aryl or heteroaryl group, optionally substituted with R³;

R¹⁰ represents R⁴, halogen, N(R⁵)₂, CN, NO₂, C(R⁶)₃, OR⁴, SR⁴ or S(O)ₙR⁷;

X represents a linker which is attached to Ar² ortho to the attachment of W, said linker containing 0-4 carbon atoms and not more than one heteroatom selected from O, N and S, said linker further optionally containing CO, S(O)n, C═C or an acetylenic group, and said linker being optionally substituted with R¹¹;

R¹¹ is R⁹;

Q represents a member selected from the group consisting of: CO₂H, tetrazole, SO₃H, hydroxamic acid, CONHSO₂R¹² and SO₂NHCOR¹²;

R¹² represents a member selected from the group consisting of: CF₃, lower alkyl, lower alkenyl, lower alkynyl and ZAr⁴, wherein Z is an optional linker containing 0-4 carbon atoms, optionally substituted with R¹³;

R¹³ is R⁹;

Ar⁴ is an aryl or heteroaryl group optionally substituted with R¹⁴, and

R¹⁴ is R¹⁰ or NHCOMe.

The compounds above can be synthesized in accordance with the following general instructions and reaction schemes:

Method A

An aryl alkene I can be coupled with an aryl bromide, iodide or triflate II in the presence of a catalyst such as Pd(OAc)2 to give the two isomers III and IV. Catalytic hydrogenation of the double bond, using Pd/C or (Ph₃P)₃RhCl, yield the compound VI. Alternatively, VI can be prepared from I via formation of the boronate V with 9-borabicyclo[3.3.1]nonane and coupling with II in the presence of a catalyst such as PdCl₂(dppf). Cyclopropanation of the alkenes III and IV can be performed using conditions such as CH₂N₂/PdOAc₂ to give VII and VIII. The group X-Q in compounds III, IV, VI, VII and VIII can then be transformed to another X-Q group to afford other substructures of II.

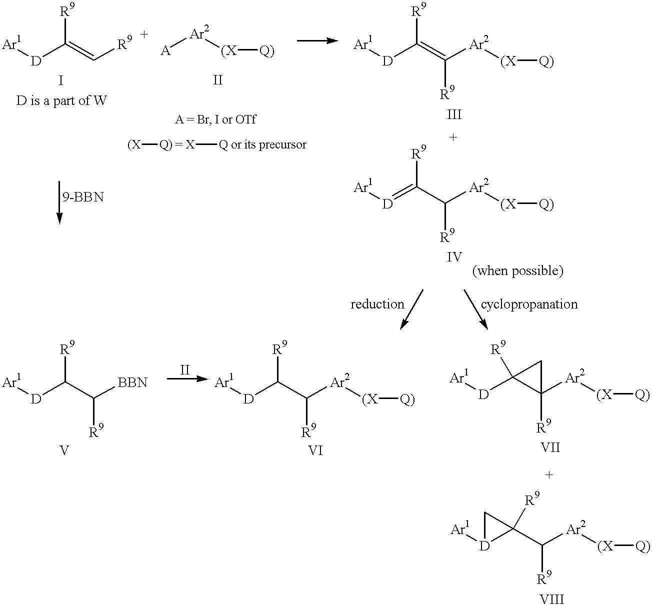

Method B

The acid or esters IX can be reduced to the alcohol X using reagents such as diisobutylaluminum hydride or sodium borohydride. Oxidation to the aldehyde XI can be performed using MnO₂ or pyridinium chlorochromate. Wittig reaction on XI afford the propenoate XII which can be cyclopropanated (CH₂N₂/Pd(OAc)₂) to XIII or reduced (H₂/Pd/C) to XIV. When R═H, compounds IX, XII, XIII and XIV are substructures of II.

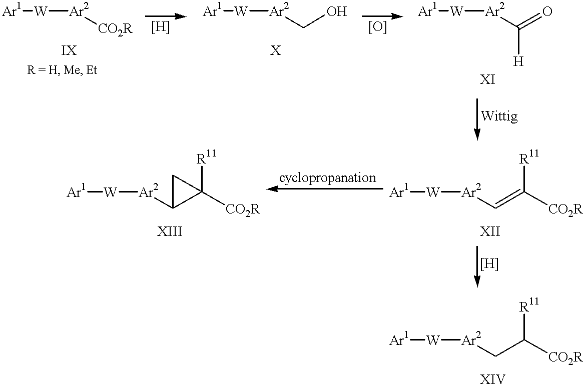

Method C

The acid XV, which is a substructure of II, can be transformed to the sulfonamide XVI, another substructure of II, by treatment with a sulfonamine in the presence of a coupling reagent such as 1-(3-dimethylaminopropyl)-3-ethylcarbodiimide. Another method for the preparation of XVI involves the formation of an acid chloride or a mixed anhydride XVII and reaction with the sulfonamine in the presence of a base such as Et₃N.

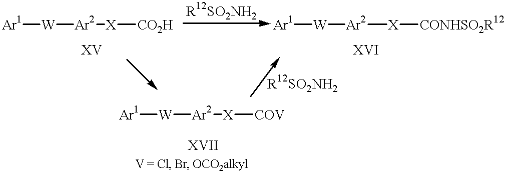

Method D

When compound II or its precursor is substituted by an hydroxyl group as in XVIII, it can be alkylated by a reagent containing a leaving group XIX in the presence of a base such as NaH or DBU to yield the ether XX. Alternatively, Mitsunobu reaction with the alcohol derivative of XIX also yield XX. The group X-Q in XX can then be transformed to another X-Q group to afford another example of II.

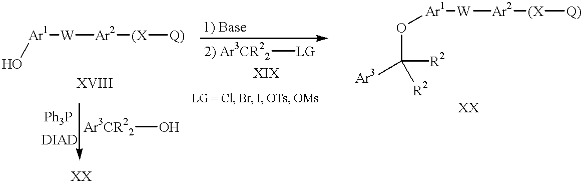

Method E

The aryl bromide, iodide or triflate XXI can be coupled with an alkyne or the alkene XXIII in the presence of a catalyst such as Pd(OAc)2 (J. Org. Chem. 1979, 4078) to give the products XXII or XXIV respectively. Catalytic hydrogenation of the alkyne XMI over Lindlar's catalyst can afford the cis alkene XXV. When R ═H, compounds XXII, XXIV and XXV are substructures of II and they can be treated as in method B to yield other examples of II.

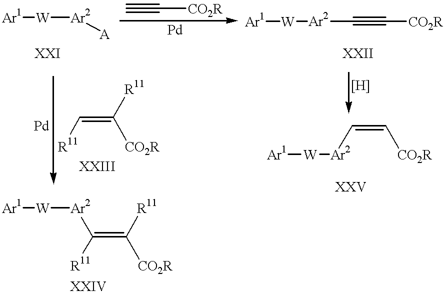

Method F

An aryl thiol, alcohol or amine XXVI can be treated with a base and then with reagent XXVII to yield the derivative XXVIII. The group E′-F-Q can be transformed to another E′-F-Q group using the other methods described here and yield examples of II possessing an heteroatom attached to Ar² in the linker X.

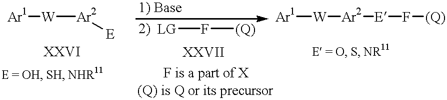

Method G

Compounds II possessing a cyclopropane unit as an X group XXX can be synthesized via a reaction between the alkene XXIX and a diazoacetate in the presence of a catalyst such as rhodium acetate dimer.

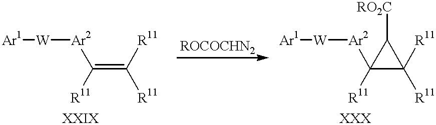

Method H

Compounds II possessing a double bond as part of the linker X can be synthesized via a Wittig reaction as exemplified in the next scheme. Phosphonium salts XXII and XXXIV can be obtained from the corresponding Ar-CHR⁹-LG by reaction with Ph₃P.

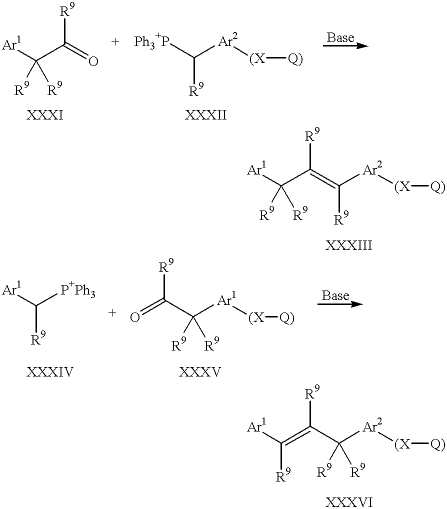

Method I

Compounds II possessing two heteroatoms as part of the linker W as in XL can be synthesized from a reagent containing two leaving groups XXVII and two aromatics compounds containing an alcohol, an amine or a thiol function E as described in the following scheme.

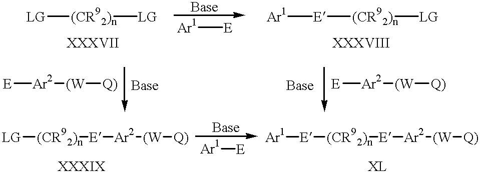

Method J

Compounds II possessing one heteroatom as part of the linker W as in XLV can be synthesized from a reagent containing one leaving group XLII or XLIII and an aromatic compound containing an alcohol, an amine or a thiol function E (XLI or XLIV) as described in the following two equations.

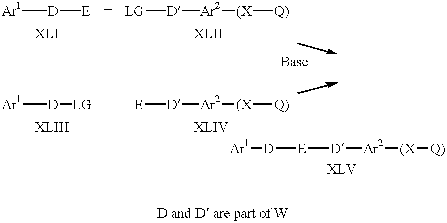

Examples of such compounds are the following:

TABLE I
(Ar¹-W-Ar²-X-Q)

| Ex. | Ar¹ | W | Ar² | X | Q |
|---|---|---|---|---|---|
| 1 | 2-(BnO)-3- | (CH₂)₃ | 1,2-Phe | (CH₂)₂ | CO₂H |
| | MePh | | | | |
| 2 | 2-(BnO)-3- | CH₂CH═CH | 1,2-Phe | CH═CH | CONHSO₂- |
| | MePh | | | | 2-thien 1 |
| 3 | 2-(BnO)-3- | CH═CHCH₂ | 1,2-Phe | CH═CH | CONHSO₂-2- |
| | MePh | | | | thienyl |
| 4 | 2-((2-Cl-4-FPh) | CH₂CH═CH | 1,2-Phe CHCH | CO₂H | |
| | CH₂O)-3- | | | | |
| | CF₃Ph | | | | |
| 5 | 2-((2-Cl-4-FPh) | CH═CHCH₂ | 1,2-Phe | CHCH | CO₂H |
| | CH₂O)-3- | | | | |
| | CF₃Ph | | | | |
| 6 | 2-(BnO)Ph | CH₂CH═CH | 1,2-Phe | CH═CH | CO₂Na |
| 7 | 2-(BnO)Ph | CH═CHCH₂ | 1,2-Phe | CH═CH | CO₂Na |
| 8 | 4-(BnO)-3,5- | CH₂CH═CH | 1,2-Phe | CH═CH | CO₂Na |
| | (MeO)₂Ph | | | | |
| 9 | 4-(BnO)-3,5- | CH═CHCH₂ | 1,2-Phe | CH═CH | CO₂Na |
| | (MeO)₂Ph | | | | |
| 10 | 2-(BnO)-5- | CH₂CH═CH | 1,2-Phe | CH═CH | CO₂H |
| | AcPh | | | | |
| 11 | 2-(BnO)-5- | CH═CHCH₂ | 1,2-Phe | CH═CH | CO₂H |
| | AcPh | | | | |
| 12 | 2-(BnO)-3- | CH₂CH═CH | 1,2-Phe | CH═CH | CO₂Na |
| | (MeO)Ph | | | | |
| 13 | 2-(BnO)-3- | CH═CHCH₂ | 1,2-Phe | CH═CH | CO₂Na |
| | (MeO)Ph | | | | |
| 14 | 4-(BnO)-3- | CH₂CH═CH | 1,2-Phe | CH═CH | CO₂Na |
| | (MeO)Ph | | | | |
| 15 | 4-(BnO)-3- | CH═CHCH₂ | 1,2-Phe | CH═CH | CO₂Na |
| | (MeO)Ph | | | | |
| 16 | 2-(BnO O)-3- | CH₂CH═CH | 1,2-Phe | CH₂ | CO₂Na |
| | MePh | | | | |
| 17 | 2-(BnO)-3- | CH═CHCH₂ | 1,2-Phe | CH₂ | CO₂Na |
| | MePh | | | | |
| 18 | 2-(BnO)-3- | CH₂CH═CH | 5-Cl-1,2- | CH₂ | CO₂Na |
| | MePh | | Phe | | |
| 19 | 2-(BnO)-3- | CH═CHCH₂ | 5-Cl-1,2- | CH₂ | CO₂Na |
| | MePh | | Phe | | |
| 20 | 4-(BnO)-3- | (CH₂)₃ | 1,2-Phe | 1,2-c-Pr | CO₂H |
| | (MeO)Ph | | | | |
| 21 | 2-(BnO)-3- | CH═CHCH₂ | 4,5-(MeO)₂- | CH═CH | CO₂H |
| | MePh | | 1,2-Phe | | |
| 22 | 2-(BnO)-3- | CH₂CH═CH | 4,5-(MeO)₂- | CH═CH | CO₂H |
| | MePh | | 1,2-Phe | | |
| 23 | 3,4-(methylene | CH═CHCH₂ | 1,2-Phe | CH═CH | CO₂H |
| | dioxy)Ph | | | | |
| 24 | 3,4-(methylene | CH₂CH═CH | 1,2-Phe | CH═CH | CO₂H |
| | dioxy)Ph | | | | |
| 25 | Ph | CH═CHCH₂ | 1,2-Phe | CH═CH | CO₂H |
| 26 | Ph | CH₂CH═CH | 1,2-Phe | CH═CH | CO₂H |
| 27 | 2-(HO)-3-MePh | CH═CHCH₂ | 1,2-Phe | CH═CH | CO₂H |
| 28 | 2-(BnO)-3- | CH═CHCH₂ | 1,2-Phe | CH═CH | CO₂Na |
| | MePh | | | | |
| 29 | 2-(BnO)-3- | CH₂CH═CH | 1,2-Phe | CH═CH | CO₂Na |
| | MePh | | | | |
| 30 | 2-((7-Cl-2- | CH═CHCH₂ | 1,2-Phe | CH═CH | CO₂H |
| | quinolinyl)CH₂O)- | | | | |
| | 3-MePh | | | | |
| 31 | 2-((7-Cl-2- | CH₂CH═CH | 1,2-Phe | CH═CH | CO₂H |
| | quinolinyl)CH₂O)- | | | | |
| | 3-MePh | | | | |
| 32 | 2-(BnO)-3- | (CH₂)₃ | 1,2-Phe | bond | CO₂H |
| | MePh | | | | |
| 33 | 2-(BnO)-3- | CH═CHCH₂ | 1,2-Phe | bond | CO₂Na |
| | MePh | | | | |
| 34 | 2-(BnO)-3- | CH₂CH═CH | 1,2-Phe | bond | CO₂Na |
| | MePh | | | | |
| 35 | 2-(BnO)-3- | (CH₂)₃ | 1,2-Phe | CH═CH | CO₂Na |
| | MePh | | | | |
| 36 | 2-(BnO)-3- | (CH₂)₃ | 1,2-Phe | CH═CH | CO₂Na |
| | (MeO)Ph | | | | |
| 37 | 4-(BnO)-3- | (CH₂)₃ | 1,2-Phe | CH═CH | CO₂Na |
| | (MeO)Ph | | | | |
| 38 | 4-(MeO)Ph | CH₂CH═CH | 1,2-Phe | CH═CH | CO₂H |
| 39 | 4-(MeO)Ph | CH═CHCH₂ | 1,2-Phe | CH═CH | CO₂H |
| 40 | 3,4-(MeO)₂Ph | CH₂CH═CH | 1,2-Phe | CH═CH | CO₂H |
| 41 | 3,4-(MeO)₂Ph | CH═CHCH₂ | 1,2-Phe | CH═CH | CO₂H |
| 42 | 2-(BnO)Ph | CH(OH)CH | 1,2-Phe | CH═CH | CO₂Na |
| | | ═CH | | | |
| 43 | 2-(BnO)-3- | (CH₂)₃ | 1,2-Phe | (CH₂)₂ | CONNaSO₂-2- |
| | MePh | | | | thienyl |
| 44 | 4-(BnO)-3- | CH₂CHCH | 1,2-Phe | CH═CH | CONNaSO₂-2- |
| | (MeO)Ph | | | | thienyl |
| 45 | 4-(BnO)-3- | CH═CHCH₂ | 1,2-Phe | CH═CH | CONNaSO₂-2- |
| | (MeO)Ph | | | | thienyl |
| 46 | 2-(BnO)-3- | CH₂-1,2-c-Pr | 1,2-Phe | CH═CH | CO₂Na |
| | MePh | | | | |
| 47 | 2-(BnO)-3- | 1,2-c-Pr-CH₂ | 1,2-Phe | CH═CH | CO₂Na |
| | MePh | | | | |
| 48 | 2-(BnO)-3- | CH(OH)CH | 1,2-Phe | CH═CH | CO₂Na |
| | MePh | ═CH | | | |
| 49 | 2-(BnO)-3- | CH═CHCH | 1,2-Phe | CH═CH | CO₂H |
| | MePh | (OH) | | | |
| 50 | 2-((2,6-Cl₂Ph) | CH═CHCH | 1,2-Phe | CH═CH | CO₂H |
| | CH₂O)-3-MePh | (OH) | | | |
| 51 | 2-((2,6-Cl2Ph) | CH(OH)CH | 1,2-Phe | CH═CH | CO₂H |
| | CH₂O)-3-MePh | ═CH | | | |
| 52 | 2-((4-FPh) | CH₂CH═CH | 1,2-Phe | CH═CH | CO₂H |
| | CH₂O)-3-MePh | | | | |
| 53 | 2-((4-FPh) | CH═CHCH₂ | 1,2-Phe | CH═CH | CO₂Na |
| | CH₂O)-3-MePh | | | | |
| 54 | 2-((3,4-F₂Ph) | CH₂CH═CH | 1,2-Phe | CH═CH | CO₂H |
| | CH₂O)-3-MePh | | | | |
| 55 | 2-((3,4-F₂Ph) | CH═CHCH₂ | 1,2-Phe | CH═CH | CO₂Na |
| | CH₂O)-3-MePh | | | | |
| 56 | 2-((3,5-F₂Ph) | CH₂CH═CH | 1,2-Phe | CH═CH | CO₂H |
| | CH₂O)-3-MePh | | | | |
| 57 | 2-((3,5-F₂Ph) | CH═CHCH₂ | 1,2-Phe | CH═CH | CO₂Na |
| | CH₂O)-3-MePh | | | | |
| 58 | 2-((2,6-Cl₂Ph) | CH₂CH═CH | 1,2-Phe | CH═CH | CO₂H |
| | CH₂O)-3- | | | | |
| | (HOCH₂)Ph | | | | |
| 59 | 2-((2,6-Cl₂Ph) | CH═CHCH₂ | 1,2-Phe | CH═CH | CO₂H |
| | CH₂O)-3- | | | | |
| | (HOCH₂)Ph | | | | |
| 60 | 2-((2,6-Cl₂Ph) | CH₂CH═CH | 1,2-Phe | CH═CH | CO₂H |
| | CH₂O)-3-Me Ph | | | | |
| 61 | 2-((2,6-Cl₂Ph) | CH═CHCH₂ | 1,2-Phe | CH═CH | CO₂Na |
| | CH₂O)-3-MePh | | | | |
| 62 | 2-((4-CF₃Ph) | CH₂CH═CH | 1,2-Phe | CH═CH | CO₂H |
| | CH₂O)-3-MePh | | | | |
| 63 | 2-((4-CF₃Ph) | CH═CHCH₂ | 1,2-Phe | CH═CH | CO₂Na |
| | CH₂O)-3-MePh | | | | |
| 64 | 2-((4- | CH₂CH═CH | 1,2-Phe | CH═CH | CO₂H |
| | (CHF₂O)Ph) | | | | |
| | CH₂O)-3-MePh | | | | |
| 65 | 2-((4-(CHF₂O) | CH═CHCH₂ | 1,2-Phe | CH═CH | CO₂Na |
| | Ph) CH₂O)-3- | | | | |
| | MePh | | | | |
| 66 | 2-((4-CF₃Ph) | CH═CHCH | 1,2-Phe | CH═CH | CO₂H |
| | CH₂O)-3- | (OH) | | | |
| | (HOCH₂)Ph | | | | |
| 67 | 2-((4-CF₃Ph) | CH═CHCH₂ | 1,2-Phe | CH═CH | CO₂H |
| | CH₂O)-3- | | | | |
| | (HOCH₂)Ph | | | | |
| 68 | 2-((4-CF₃Ph) | CH═CHCH | 1,2-Phe | CH═CH | CO₂H |
| | CH₂O)-3-MePh | (OH) | | | |
| 69 | 2-(PhCH₂O)-3- | CH═CHCH₂ | 1,2-Phe | CH═CH | CO₂H |
| | (HOCH₂)Ph | | | | |
| 70 | 3-(PhO)Ph | CH₂OCH₂ | 1,2-Phe | CH═CH | CO₂Na |
| 71 | 2-(PhO)Ph | CH₂OCH₂ | 1,2-Phe | CH═CH | CO₂Na |
| 72 | 3-(BnO)Ph | CH₂CH═CH | 1,2-Phe | CH═CH | CO₂Na |
| 73 | 3-(BnO)Ph | CH═CHCH₂ | 1,2-Phe | CH═CH | CO₂Na |
| 74 | 2-(BnO)Ph | O(CH₂)₃O | 1,2-Phe | CH═CH | CO₂Na |
| 75 | 2-(PhCHMeO)- | CH═CHCH₂ | 1,2-Phe | CH═CH | CO₂Na |
| | 3- | | | | |
| | MePh | | | | |
| 76 | 2-(PhCHMeO)- | CH₂CH═CH | 1,2-Phe | CH═CH | CO₂H |
| | 3- | | | | |
| | MePh | | | | |
| 77 | 3-(PhO)Ph | CH═CHCH₂ | 1,2-Phe | CH═CH | CO₂Na |
| 78 | 3-(PhO)Ph | CH₂CH═CH | 1,2-Phe | CH═CH | CO₂Na |
| 79 | 3-Ph | CH═CHCH₂ | 1,2-Phe | CH═CH | CO₂Na |
| | benzofuran-7- | | | | |
| 80 | 3-Ph | CH₂CH═CH | 1,2-Phe | CH═CH | CO₂Na |
| | benzofuran-7- | | | | |
| 81 | Ph | CH═CHCH₂ | 1,2-Phe | CH═CH | CONHSO₂- |
| | | | | | 2-thienyl |
| 82 | Ph | CH₂CH═CH | 1,2-Phe | CH═CH | CONHSO₂- |
| | | | | | 2-thienyl |
| 83 | 4-(MeO)Ph | CH═CHCH₂ | 1,2-Phe | CH═CH | CONHSO₂-2- |
| | | | | | thienyl |
| 84 | 4-(MeO)Ph | CH₂CH═CH | 1,2-Phe | CH═CH | CONHSO₂-2- |
| | | | | | thienyl |
| 85 | 2-(BnO)-1- | CH₂NHCO | 1,2-Phe | CH═CH | CO₂H |
| | naphthyl | | | | |
| 86 | 2-((2-Cl-4-FPh) | CH₂CH═CH | 1,2-Phe | CH═CH | CO₂H |
| | CH₂O)-3-MePh | | | | |
| 87 | 2-((2-Cl-4-FPh) | CH═CHCH₂ | 1,2-Phe | CH═CH | CO₂H |
| | CH₂O)-3-MePh | | | | |
| 88 | 2-((2,4-F₂Ph) | CH₂CH═CH | 1,2-Phe | CH═CH | CO₂H |
| | CH₂O)-3-MePh | | | | |
| 89 | 2-((2,4-F₂Ph) | CH═CHCH₂ | 1,2-Phe | CH═CH | CO₂H |
| | CH₂O)-3-MePh | | | | |
| 90 | 2-((2,4,6-F₃Ph) | CH₂CH═CH | 1,2-Phe | CH═CH | CO₂H |
| | CH₂O)-3-MePh | | | | |
| 91 | 2-((2,4,6-F₃Ph) | CH═CHCH₂ | 1,2-Phe | CH═CH | CO₂H |
| | CH₂O)-3-MePh | | | | |
| 92 | 2-((2,6-Cl₂-4- | CH₂CH═CH | 1,2-Phe | CH═CH | CO₂H |
| | FPh) | | | | |
| | CH₂O)-3-MePh | | | | |
| 93 | 2-((2,6-Cl₂-4- | CH═CHCH₂ | 1,2-Phe | CH═CH | CO₂H |
| | FPh) | | | | |
| | CH₂O)-3-MePh | | | | |
| 94 | 2-((2,4- | CH₂CH═CH | 1,2-Phe | CH═CH | CO₂H |
| | F₂Ph) CH₂O) | | | | |
| | -3-(CHF₂O)Ph | | | | |
| 95 | 2-((2,4-F₂Ph) | CH═CHCH₂ | 1,2-Phe | CH═CH | CO₂H |
| | CH₂O) | | | | |
| | -3-(CHF₂O)Ph | | | | |
| 96 | 2-((4- | CF₂CH═CH | 1,2-Phe | CH═CH | CO₂H |
| | FPh)CH₂O) | | | | |
| | -3-MePh | | | | |
| 97 | 2-((4- | CH═CHCF₂ | 1,2-Phe | CH═CH | CO₂H |
| | FPh)CH₂O) | | | | |
| | -3-MePh | | | | |
| 98 | 2-((4- | (CH₂)₃ | 1,2-Phe | CH═CH | CONHSO₂-(4- |
| | FPh) CH₂O) | | | | i-PrPh) |
| | -3-MePh | | | | |
| 99 | 2-((4- | (CH₂)₃ | 1,2-Phe | CH═CH | CONHSO₂-(4- |
| | FPh) CH₂O) | | | | t-BuPh) |
| | -3-MePh | | | | |
| 100 | 2-((4- | CH₂CH═CH | 1,2-Phe | CH═CH | CONHSO₂-(4- |
| | FPh) CH₂O) | | | | (MeO)Ph) |
| | -3-MePh | | | | |
| 101 | 2-((4- | CH═CHCH₂ | 1,2-Phe | CH═CH | CONHSO₂- |
| | FPh) CH₂O) | | | | (2,3-Cl₂Ph) |
| | -3-MePh | | | | |
| 102 | 2-((4- | CH═CHCH₂ | 4-Cl-1,2- | CH═CH | CONHSO₂-(5- |
| | FPh) CH₂O) | | Phe | | Br-2- |
| | -3-MePh | | | | (MeO)Ph) |
| 103 | 2-((4- | (CH₂)₂S | 3-F-1,2-Phe | CH═CH | CONHSO₂- |
| | FPh) CH₂O) | | | | (2,3,4-Cl₃Ph) |
| | -3-MePh | | | | |
| 104 | 2-((4-FPh) | (CH₂)₂S | 6-CF₃-1,2- | CH═CH | CONHSO₂-(5- |
| | CH₂O)-3-MePh | | Phe | | F-2-MePh) |
| 105 | 2-((4-FPh) | (CH₂)₂S | 4,5-F₂-1,2- | CH═CH | CONHSO₂- |
| | CH₂O)-3-MePh | | Ph | | (2,5-Me₂Ph) |
| 106 | 2-((4-FPh) | (CH₂)₂SO₂ | 1,2-Phe | CH═CH | CONHSO₂-(4- |
| | CH₂O)-3-MePh | | | | CF₃Ph) |
| 107 | 2-((4-FPh) | (CH₂)₂SO₂ | 1,2-Phe | CH═CH | CONHSO₂-2- |
| | CH₂O)-3-MePh | | | | naphthyl |
| 108 | 2-((4-FPh) | CH═CHCH₂ | 3-F-1,2-Phe | CH═CH | CONHSO₂-(3- |
| | CH₂O)-3-MePh | | | | Cl-4-FPh) |
| 109 | 2-((4-FPh) | SO₂(CH₂)₂ | 1,2-Phe | CH═CH | CONHSO₂-(4- |
| | CH₂O)-3-MePh | | | | n-PrPh) |
| 110 | 2-((4-FPh) | SO₂(CH₂)₂ | 1,2-Phe | CH═CH | CONHSO₂-(2- |
| | CH₂O)-3- | | | | ClPh) |
| | (MeO)Ph | | | | |
| 111 | 2-((4-FPh) | SO₂(CH₂)₂ | 1,2-Phe | CH═CH | CONHSO₂-(4- |
| | CH₂O)-3- | | | | FPh) |
| | (MeO)Ph | | | | |
| 112 | 2-((4-FPh) | S(CH₂)₂ | 1,2-Phe | CH═CH | CONHSO₂-(2- |
| | CH₂O)-3- | | | | PhPh) |
| | (MeO)Ph | | | | |
| 113 | 2-((4-FPh) | S(CH₂)₂ | 1,2-Phe | CH═CH | CONHSO₂-(2- |
| | CH₂O)-3- | | | | CF₃Ph) |
| | (MeO)Ph | | | | |
| 114 | 2-((4-FPh) | S(CH₂)₂ | 4-t-Bu-1,2- | CH═CH | CONHSO₂-(4- |
| | CH₂O)-3- | | Phe | | Cl-2,5-Me₂Ph) |
| | (MeO)Ph | | | | |
| 115 | 2-((4-FPh) | O(CH₂)₂ | 1,2-Phe | CH═CH | CONHSO₂- |
| | CH₂O)-3- | | | | (2,5-Cl₂Ph) |
| | (MeO)Ph | | | | |
| 116 | 2-((4-FPh) | O(CH₂)₂ | 1,2-Phe | CH═CH | CONHSO₂-(4- |
| | CH₂O)-3- | | | | Br-2- |
| | (MeO)Ph | | | | (CF₃O)Ph) |
| 117 | 2-((4-FPh) | O(CH₂)₂ | 1,2-Phe | CH═CH | CONHSO₂- |
| | CH₂O)-3- | | | | CH₂Ph |
| | (MeO)Ph | | | | |
| 118 | 2-((4-FPh) | (CH₂)₂O | 1,2-Phe | CH═CH | CONHSO₂-1- |
| | CH₂O)-3- | | | | naphthyl |
| | (MeO)Ph | | | | |
| 119 | 2-((4-FPh) | (CH₂)₂O | 4,5-F₂-1,2- | CH═CH | CONHSO₂-(2- |
| | CH₂O)-3- | | Phe | | FPh) |
| | (MeO)Ph | | | | |
| 120 | 2-((4-FPh) | (CH₂)₂O | 1,2-Phe | CH═CH | CONHSO₂- |
| | CH₂O)-3- | | | | (2,4-Cl₂Ph) |
| | (MeO)Ph | | | | |
| 121 | 2-((4-FPh) | (CH₂)₃ | 1,2-Phe | CH═CH | CONHSO₂- |
| | CH₂O)-3- | | | | CH═CHPh |
| | (MeO)Ph | | | | |
| 122 | 2-((4- | (CH₂)₃ | 1,2-Phe | CH═CH | CONHSO₂- |
| | FPh) CH₂O)-3- | | | | (3,5- |
| | (MeO)Ph | | | | (CF₃)₂Ph) |
| 123 | 2-((4- | (CH₂)₃ | 1,2-Phe | CH═CH | CONHSO₂- |
| | FPh)CH₂O)Ph | | | | (2,5-Cl₂-3- |
| | | | | | thienyl) |
| 124 | 2-((4-FPh) | (CH₂)₄ | 3-F-1,2-Phe | CH═CH | CONHSO₂-(3- |
| | CH₂O)Ph | | | | BrPh) |
| 125 | 2-((4-FPh) | (CH₂)₄ | 3-MeO-1,2- | CH═CH | CONHSO₂-(2- |
| | CH₂O)Ph | | Phe | | BrPh) |
| 126 | 2-((4-FPh) | (CH₂)₄ | 1,2-Phe | CH═CH | CONHSO₂-(2- |
| | CH₂O)Ph | | | | NO₂Ph) |
| 127 | 2-((4-FPh) | (CH₂)₅ | 1,2-Phe | (CH₂)₂ | CONHSO₂-(3- |
| | CH₂O)Ph | | | | ClPh) |
| 128 | 2-((4-FPh) | (CH₂)₅ | 1,2-Phe | (CH₂)₂ | CONHSO₂-(4- |
| | CH₂O)Ph | | | | (CF₃O)Ph) |
| 129 | 2-HOPh | CH═CH(CH₂)₂ | 1,2-Phe | (CH₂)₂ | CONHSO₂-8- |
| | | | | | quinolinyl |
| 130 | 2-((4-FPh) | CH═CH(CH₂)₂ | 5-(CF₃O)- | (CH₂)₂ | CONHSO₂- |
| | CH₂O)Ph | | 1,2-Phe | | (3,4-Cl₂Ph) |
| 131 | 4-((2,6-Cl₂-4- | CH═CH(CH₂)₂ | 3-F-1,2-Phe | (CH₂)₂ | CONHSO₂-(4- |
| | FPh)CH₂O)-3- | | | | EtPh) |
| | MePh | | | | |
| 132 | 2-((4-FPh) | CH₂CH═CH | 1,2-Phe | (CH₂)₂ | CONHSO₂-(4- |
| | CH₂O)Ph | | | | Cl-2-NO₂Ph) |
| 133 | 2-((4-FPh) | CH═CHCH₂ | 4,5-F₂-1,2- | CH═CH | CONHSO₂-(2- |
| | CH₂O)Ph | | Phe | | Cl-3-Br-5- |
| | | | | | thienyl) |
| 134 | 2-((4-FPh) | CH₂CH═CH | 4,5-F₂-1,2- | CH═CH | CONHSO₂- |
| | CH₂O)Ph | | Phe | | (3,4- |
| | | | | | (MeO)₂Ph) |
| 135 | 2-HOPh | CH═CHCH₂ | 4,5-F₂-1,2- | CH═CH | CONHSO₂- |
| | | | Phe | | (2,5-Cl₂-3-Br- |
| | | | | | 4-thienyl) |
| 136 | 4-((4- | CH₂CH═CH | 4,5-F₂-1,2- | CH═CH | CONHSO₂-(4- |
| | FPh)CH₂O)- | | Phe | | Br-2,5-F₂Ph) |
| | 3-(MeO)Ph | | | | |
| 137 | 4-((4- | CH═CHCH₂ | 1,2-Phe | CH═CH | CONHSO₂-(5- |
| | FPh)CH₂O)- | | | | (AcNH)-1,3,4- |
| | 3-(MeO)Ph | | | | thiadiazol-2- |
| 138 | 4-((4-FPh) | CH₂CH═CH | 1,2-Phe | CH═CH | CONHSO₂- |
| | CH₂O)-3- | | | | (2,3,4,5,6- |
| | (MeO)Ph | | | | F₅Ph) |
| 139 | 4-((2-Cl-4-FPh) | CH═CHCH₂ | 1,2-Phe | CH═CH | CONHSO₂-(2- |
| | CH₂O)-3-MePh | | | | CNPh) |
| 140 | 2-((4-FPh) | CH₂CH═CH | 4-F-1,2-Phe | CH═CH | CONHSO₂-(2- |
| | CH₂O)Ph | | | | Cl-6-Me Ph) |
| 141 | 2-HOPh | CH═CHCH₂ | 1,2-Phe | CH═CH | CONHSO₂- |
| | | | | | (2,4,6-Me₃Ph) |
| 142 | Ph | CH₂CH═CH | 1,2-Phe | CH═CH | CONHSO₂- |
| | | | | | (2,3-Br₂-2- |
| | | | | | thienyl) |
| 143 | 2-((4-FPh) | CH═CHCH₂ | 1,2-Phe | CH₂O | CONHSO₂-(4- |
| | CH₂O)Ph | | | | NO₂Ph) |
| 144 | 2-((4-FPh) | CH₂CH═CH | 1,2-Phe | CH₂O | CONHSO₂- |
| | CH₂O)Ph | | | | (3,5-Cl₂Ph) |
| 145 | 2,4-((4-FPh) | CH═CHCH₂ | 1,2-Phe | prop-1- | CONHSO₂-(5- |
| | CH₂O)₂Ph | | | yne-1,3-Cl- | 2-thienyl) |
| | | | | diyl | |
| 146 | 4-((2,4-F₂Ph) | CH₂CH═CH | 1,2-Phe | CH₂O | CONHSO₂-(4- |
| | CH₂O)-3- | | | | CF₃Ph) |
| | (MeO)Ph | | | | |
| 147 | 2-HO-3-MePh | CH═CHCH₂ | 1,2-Phe | CH₂O | CONHSO₂- |
| | | | | | (2,4-F₂Ph) |
| 148 | 2-((4-FPh) | CH₂CH═CH | 4-F-1,2-Phe | 1,2-ethyne | CONHSO₂-(4- |
| | CH₂O)Ph | | | diyl | ClPh) |
| 149 | 2-((4-FPh) | CH═CHCH₂ | 1,2-Phe | 1,2-ethyne | CONHSO₂-(3- |
| | CH₂O)Ph | | | diyl | CF₃Ph) |
| 150 | 4-HOPh | CH₂CH═CH | 1,2-Phe | 1,2-ethyne | CONHSO₂-Ph |
| | | | | diyl | |
| 151 | 2-((4-FPh) | CH═CHCH₂ | 1,2-Phe | prop-2- | CONHSO₂-(5- |
| | CH₂O)Ph | | | yne-1,3- | Br-2-thienyl) |
| | | | | diyl | |
| 152 | 2,4-((4-FPh) | CH₂CH═CH | 1,2-Phe | 1,2- | CONHSO₂- |
| | CH₂O)₂Ph | | | ethynediyl | Me |
| 153 | 2,4-((4-FPh) | CH═CHCH₂ | 1,2-Phe | 1,2-c-Pr | CONHSO₂- |
| | CH₂O)₂Ph | | | | (2,5- |
| | | | | | (MeO)₂Ph) |
| 154 | 6-((4-FPh) | CH₂CH═CH | 4-F-1,2-Phe | 1,2-c-Pr | CONHSO₂-(3- |
| | CH₂O)-2- | | | | MePh) |
| | naphthyl | | | | |
| 155 | 2-((4-FPh) | CH═CHCH₂ | 1,2-Phe | 1,2-c-Pr | CONHSO₂-(4- |
| | CH₂O)Ph | | | | MePh) |
| 156 | 4-HO-3- | CH₂CH═CH | 1,2-Phe | 1,2-c-Pr | CONHSO₂-n- |
| | (Me0)Ph | | | | Bu |
| 157 | 4-((4- | CH═CHCH₂ | 1,2-Phe | 1,2-c-Bu | CONHSO₂-(2- |
| | FPh)CH₂O)- | | | | Cl-4-FPh) |
| | 1-naphthyl | | | | |
| 158 | Ph | CH₂CH═CH | 1,2-Phe | CH═CH | CONHSO₂- |
| | | | | | (2-MePh) |
| 159 | 2-((4-FPh) | CH═CHCH₂ | 1,2-Phe | CH═CH | CONHSO₂- |
| | CH₂O)Ph | | | | c-Pr |
| 160 | 2,4-((4-FPh) | CH═CHCH₂ | 1,2-Phe | CH═CH | CO₂H |
| | CH₂O)₂Ph | | | | |
| 161 | 4-((2,4-F₂Ph) | (CH₂)₃ | 4-F-1,2-Phe | CH═CH | 1H-tetrazol- |
| | CH₂O)-3- | | | | 5-yl |
| | (MeO)Ph | | | | |
| 162 | 2-((4-FPh) | CH═CHCH₂ | 3-MeO- | CH═CH | 1H-tetrazol- |
| | CH₂O)Ph | | 1,2-Phe | | 5-yl |
| 163 | 2,4-((4-FPh) | CH═CHCH₂ | 1,2-Phe | CH═CH | 1H-tetrazol- |
| | CH₂O)₂Ph | | | | 5-yl |
| 164 | 4-HO-3- | CH═CHCH₂ | 1,2-Phe | 1,2-c-Pr | 1H-tetrazol- |
| | (MeO)Ph | | | | 5-yl |
| 165 | Ph | CH═CHCH₂ | 1,2-Phe | (CH₂)₂ | 1H-tetrazol- |
| | | | | | 5-yl |
| 166 | 2-((4- | CH═CHCH₂ | 1,2-Phe | CH═CH | SO₃H |
| | FPh)CH₂O)- | | | | |
| | 3-(MeO)Ph | | | | |
| 167 | 2-((4- | (CH₂)₃ | 4-F-1,2-Phe | (CH₂)₂ | SO₃H |
| | FPh)CH₂O)- | | | | |
| | 3-MePh | | | | |

Another example of E-type prostaglandin ligands can be found in U.S. application Ser. No. 60/077,990 filed on Mar. 13, 1998. Briefly, the compounds are described as falling within the following formula:

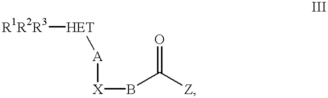

wherein:

HET represents a 5-12 membered monocyclic or bicyclic aromatic ring system containing 0-3 heteroatoms selected from O, S(O)ₙ and N(O)ₘ wherein m is 0 or 1 and n is 0, 1 or 2;

A is a one or two atom moiety and is selected from the group consisting of: —W—, —C(O)—, —C(R⁷)₂—W—, —W—C(R⁷)₂ —, —CR⁷(OR²⁰)— , —C(R⁷)₂ —, —C(R⁷)₂—C(OR²⁰)R⁷—, —C(R⁷)₂— C(R⁷)₂ or CR⁷=CR⁷, wherein W represents 0, S(O)ₙ or NR¹⁷, with n as previously defined and R¹⁷ as defined below;

X represents a 5-10 membered monocyclic or bicyclic aryl or heteroaryl group having 1-3 heteroatoms selected from O, S(O)ₙ and N(O)ₘ, and optionally substituted with R¹⁴ and R¹⁵, and A and B are attached to the aryl or heteroaryl group ortho relative to each other;

Y represents O, S(O)ₙ, NR^{17,} a bond or —CR¹⁸═CR¹⁸—;

B represents —(C(R¹⁸)₂)ₚ—Y—(C(R¹⁸)₂)_{q}—

wherein p and q are independently 0-3, such that when Y represents O, S(O)ₙ, NR¹⁷ or —CR¹⁸═CR¹⁸—, p+q=0-6, and when Y represents a bond, p+q is 1-6;

Z is OH or NHSO₂R¹⁹;

R¹ R² and R³ independently represent H, halogen, lower alkyl, lower alkenyl, lower alkynyl, lower alkenyl-HET(R^{a})₄₋₉, —(C(R⁴)₂)ₚSR⁵, —(C(R⁴)₂)ₚOR⁸, —(C(R⁴)₂)ₚN (R⁶)₂, CN NO₂, —(C(R⁴)₂)ₚC(R⁷)₃, —CO₂R⁹, —CON(R⁶)₂ or —(C(R⁴)₂)ₚS(O)ₙR¹⁰, wherein n and p are as previously defined;

each R⁴ is independently H, F, CF₃ or lower alkyl, or two R⁴ groups are taken in conjunction and represent a ring of up to six atoms, optionally containing one heteroatom selected from O, S(O), or N(O)ₘ;

each R⁵ is independently lower alkyl, lower alkenyl, lower alkynyl, CF₃, lower alkyl-HET, lower alkenyl-HET or —(C(R¹⁸)₂ )_{pPh(R}¹¹)₀₋₂;

each R⁶ is independently H, lower alkyl, lower alkenyl, lower alkynyl, CF₃, Ph, Bn and when two R⁶ groups are attached to N they may be taken in conjunction and represents a ring of up to 6 atoms, optionally containing an additional heteroatom selected from O, S(O)ₙ or N(O)ₘ;

each R⁷ is independently H, F, CF₃ or lower alkyl, and when two R⁷ groups are presents, they may be taken in conjunction and represent an aromatic or aliphatic ring of 3 to 6 members containing from 0-2 heteroatoms selected from O, S(O)ₙ and N(O)ₘ;

each R⁸ represents H or R⁵;

each R⁹ is independently H, lower alkyl, lower alkenyl, lower alkynyl, Ph or Bn;

each R¹⁰ is independently lower alkyl, lower alkenyl, lower alkynyl, CF₃, Ph(R¹¹)₀₋₃, CH₂Ph(R¹¹)₀₋₃ or N(R⁶)₂

each R¹¹ is independently lower alkyl, SR²⁰, OR²⁰, N(R⁶)₂,

—CO2R¹², —CON(R⁶)₂, —C(O)R¹², CN, CF3, NO₂ or halogen;

each R¹² is independently H, lower alkyl or benzyl;

each R¹³ is independently H, halo, lower alkyl, O-lower alkenyl, S-lower alkyl, N(R⁶)₂, CO₂R¹², CN, CF₃ or NO₂;

R¹⁴ and R¹⁵ are independently lower alkyl, halogen, CF₃, OR¹⁶, S(O)ₙR¹⁶ or C(R¹⁶)₂OR¹⁷;

each R16 is independently H, lower alkyl, lower alkenyl, Ph, Bn or CF₃;

each R¹⁷ is independently H, lower alkyl or Bn;

each R¹⁸ is independently H, F or lower alkyl, and when two R¹⁸ groups are present, they may be taken in conjunction and represent a ring of 3 to 6 members comprising carbon atoms and optionally one heteroatom chosen from O, S(O)ₙ or N;

each R¹⁹ is lower alkyl, lower alkenyl, lower alkynyl, CF₃, HET(R^{a})₄₋₉, lower alkyl-HET(R^{a})₄₋₉ or lower alkenyl-HET(Ra)₄-₉;

each R²⁰ is independently H, lower alkyl, lower alkenyl, lower alkynyl, CF₃ or Ph(R¹³)₂ and

each R^{a} is independently selected from the group consisting of: H, OH, halo, CN, NO₂, amino, C₁₋₆alkyl, C₂₋₆alkenyl, C₂₋₆alkynyl, C₁₋₆ alkoxy, C₂₋₆alkenyloxy, C₂₋₆alkynyloxy, C₁₋₆alkylamino, di-C₁₋₆alkylamino, CF₃, C(O)C₁₋₆alkyl, C(O)C₂₋₆alkenyl, C(O) C₂₋₆alkynyl, CO₂H, CO₂C₁₋₆alkyl, CO₂C_{2 6}alkenyl, and CO₂C₂-₆alkynyl,

said alkyl, alkenyl, alkynyl and the alkyl portions of alkylamino and dialkylamino being optionally substituted with 1-3 of: hydroxy, halo, aryl, C₁₋₆ alkoxy, C₂₋₆alkenyloxy, C₂₋₆alkynyloxy, CF₃, C(O)C₁₋₆alkyl, C(O)C₂₋₆alkenyl, C(O)C₂₋₆alkynyl, CO₂H, CO₂C₁₋₆alkyl, CO₂C₂₋₆alkenyl, CO₂C₂₋₆alkynyl, NH₂, NHC₁₋₆alkyl and N(C₁₋₆alkyl)₂.

The compounds noted above can be sythesized in accordance with the following general procedures and schemes.

Method A

Cinnamic ester 1 is treated with a brominating agent such as NBS in a refluxing inert solvent such as CCl₄, with the use of an initiator like benzoyl peroxide. or light. The resulting benzylic bromide is reacted in a Suzuki coupling reaction with the appropriate boronic acid or ester, a catalyst such as tetrakis(triphenylphosphine) palladium and cesium fluoride or Na₂CO₃ or a base in an inert refluxing solvent such as DME at 80-90° C. The new cinnamic ester 3 is hydrolyzed with aqueous sodium hydroxide to afford the acid 4 that is converted to the cinnamic sulfonamide 5 with a coupling reagent such as DCC or DCI in CH₂Cl₂ at r.t.

Method B

Cinnamic ester 2 is treated with a thio, hydroxy or amino aryl or heteroaryl with a base such as a hydride or an amine in benzene or THF at 0-23° C. The resulting cinnamic ester 6 is converted to 7 according to Method A.

If W=sulfur, it is oxidized to the sulfoxide or sulfone with hydrogen peroxide, m-CPBA or other peracetic acid. The cinnamic ester 9 is prepared according to Method A.

Method C

The aldehyde 11 is prepared by an addition-elimination of a thio, hydroxy or amino aryl or heteroaryl with a base such as K₂CO₃ in refluxing CHCl₃. If needed a higher boiling point solvent can be used. This type of rection can also be performed with CuO in DMF. An Emmons-Horner type reaction (or Wittig) in toluene at r.t. followed by Method A (or oxidation as described in Method B) results in the cinnamic sulfonamide 13.

Method D

Acetal 14 that came from an acetalization from a suitably substituted bromo benzaldehyde is converted to the Grignard reagent with magnesium in an etheral solvent at reflux and quenched with an aryl or heteroaryl carbonyl. The alcohol 16 is reacted with an halide and a base (or protected as the o-nitrobenzyl, and removed at the end of the sequence) to furnish the compound 17. Deprotection of the acetal under standard conditions followed by Method C and A gives 18.

Method E

Alcohol 16 is converted to an acetate with acetyl chloride (or acetic anhydride and an amine base) and coupled with a Grignard reagent and a copper salt at low temperature. The alcohol 16 could also be converted to the bromide and treated in a similar way to yield 20. Alternatively the tetrametyl acetal (R=methyl) version of alcohol 16 can be treated with TiCl₄/Me₂Zn (or R⁷₂Zn) at -30° C. Compound 20 is then converted to the cinnamic sulfonamide 21 according to Method D. Also, 22 can be treated with Al(R⁷)3 in toluene at 80° C. for 24h and 23 converted to the aldehyde with n-BuLi/DMF followed by an Emmons-Horner reaction and Method A to yield compound 21.

Method F

A suitably substituted bromo toluene 22 is treated with n-Buli at low temperature and quenched with an aryl or heteroaryl aldehyde. The resulting alcohol is oxidized to the carbonyl with PDC, PCC, MnO₂ or other typical oxidizing agent. The carbonyl is treated with SF₄, MoF₆-BF₃ (or converted to a thioacetal and treated with nitrosonium BF₄-pyridinium•HF) to yield the difluoride. Benzylic bromination with NBS followed by oxidation with N-methylmorpholine N-oxide at 100° C. in dioxane for 4 h, yielded compound 25 that is converted to cinnamic sulfonamide 26 with Method C and A.

Method G

The appropriately methyl bromo(or triflate) benzoate 27 is converted to compound 28 by a Suzuki coupling reaction followed by hydrogenation. A Stille coupling reaction could also be used. Benzylic bromination or benzylic oxidation followed by treatment with a brominating agent such as CBr₄/triphenylphosphine gives compound 29 which can be treated with a boronic acid, or a tin compound (Stille) to furnish compound 30. Reduction of the ester with DIBAL, oxidation with MnO₂ and Method C and A gives compound 31.

Method H

Compound 29 (one R⁷=H) is treated with triphenyl phosphine to give the salt and with a base such as LDA, is converted to compound 32 with the aryl or heteroaryl ketone. The halide can also be converted the Grignard reagent and added to the carbonyl. Dehydration under acidic conditions results in compound 32. Reduction of the unsaturation under standard conditions, followed by Methods G, C and A gives compound 33. From compound 32, cyclopropanation with diazomethane and palladium (0) followed by Methods G, C and A gives compound 34.

Method I

The (heterocyclic) vinylic bromide 35 is reacted in a Suzuki coupling reaction with an aryl or hetero aryl boronic acid and converted to a new boronic acid by 9-BBN addition followed by a second Suzuki reaction with compound 14. Compound 37 thus formed is reduced by hydrogenolysis (H₂/metal or diimide) and deprotection followed by Methods C and A gives cinnamic sulfonamide 39.

Method J

Ketone 40 which comes from oxidation of the corresponding alcohol is reacted with a phosphonium salt or phosphono ester with a base such as LDA to give the cinnamic ester 41. Method A yields 42 and reduction of the double bond by the previously mentioned method gives the acyl sulfonamide 43.

Method K

Cinnamic ester 3 is reduced to 44 by the previously mentioned method. α Alkylation with a base such as LDA followed by an alkylating agent results in 45 after conversion to the acyl sulfonamide.

Method L

Cinnamic ester 3 is reduced to 46 with DIBAL and the double bond converted to a cyclopropane by a Simmons-Smith reaction, or similar reactions recently described in the literature. Compound 47 is then oxidized and the cinnamic sulfonamide 48 is prepared according to Method A.

Method M

Ester 49 which can come from the homologation of the appropriately substituted methyl ortho-toluate, is treated with a base and with an alkylating agent to furnish compound 50. Benzylic bromination and Suzuki coupling gives compound 53. Homologation according to J. Amer. Chem. Soc.; 1985, 1429; J. Org. Chem. 1992, 7194, followed by alkylation with a base such as LDA and an alkylating agent furnishes acylsulfonamide 51 by Method A.

Compound 50 can also be converted to the benzylic bromide and to compound 52 by Method A.

Method N

Suitably substituted compound 53 is treated with a boronic acid to give compound 54 which is reduced with LDA to the alcohol 55. Treatment with phosgene followed with the appropriate sulfonamide gives compound 56. This can also be prepared by mixing phosgene and the sulfonamide at 140° C. to generate the isocyanate.

Compound 54 is treated with a Grignard reagent to give the corresponding alcohol and as previously described, converted to compound 57.

Method O

Ester 58 is treated with Lawesson's reagent, DAST and light to give the benzylic alcohol 59. The procedure according to Method N yields compound 60.

Method P

Compound 59 is brominated as described earlier (or iodinated) and reacted in a S_{N}2 type reaction with an ester and a base such as LDA to furnish ester 61. Method A gives the acylsulfonamide 62.

Method Q

Compound 55 is treated with NH₃/Ph₃P/DEAD (or treated with CBr₄/Ph₃P and the bromide converted to the amine 63 with ammonia). Treatment with phosgene followed by sulfonamide yields 64, treatment of which with a base and an alkyl or benzylic halide gives compounds 65.

Method R

Aldehyde 10 is treated with a silylated source of hydroxyl or thiol at 80-130° C., and the silyl group removed by fluoride treatment. Compound 66 is then treated with an aryl or heteroaryl methylene bromide with a base such as a tertiary amine in CHCl₃ or benzene to yield aldehyde 67. Emmons-Horner (or Wittig reaction) with LDA results in compound 68 via Method A.

Method S

In the case of an amine an alternative to method R can be used. A suitably substituted nitro aldehyde 69 is converted to compound 70 as described earlier and the nitro group reduced with standard methods. Mono-alkylation followed by displacement with an aryl or heteroaryl methylene bromide and processing by Method A yields cinnamic sulfonamide 71.

Method T

A suitably substituted bromo toluene 24 is converted to the anion in an etheral solvent at low temperature and trapped with an aldehyde of an aryl or heteroaryl. The resulting alcohol is oxidized with MnO2, Jones' reagent, PDC, PCC or any other oxidant. Benzylic bromination followed by oxidation with N-methyl morpholine N-oxide, yields a ketoaldehyde. Emmons-Horner and Method A gives the cinnamic sulfonamides 72.

Generic structures 4, 5, 7, 9, 13, 18, 21, 26, 31, 33, 34, 39, 42, 43, 45, 48, 51, 52, 56, 57, 60, 62, 64, 65, 68, 71 and 72 are representative of the compounds used in the present invention. It is also noted that where the chemistry allows in the generic schemes, alternate embodiments of -A-, such as heteroaryl groups, can be substituted for phenyl in the schemes.

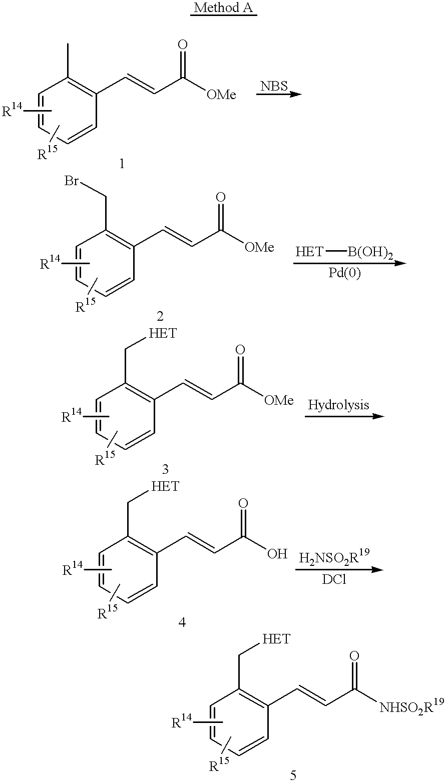

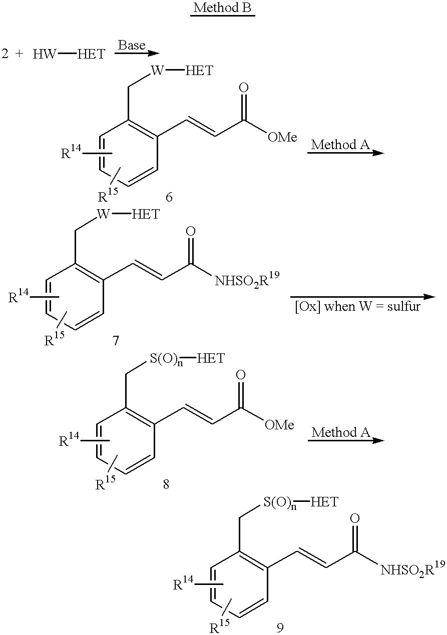

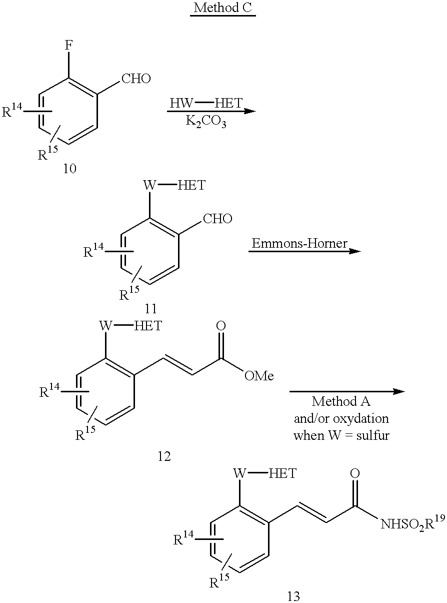

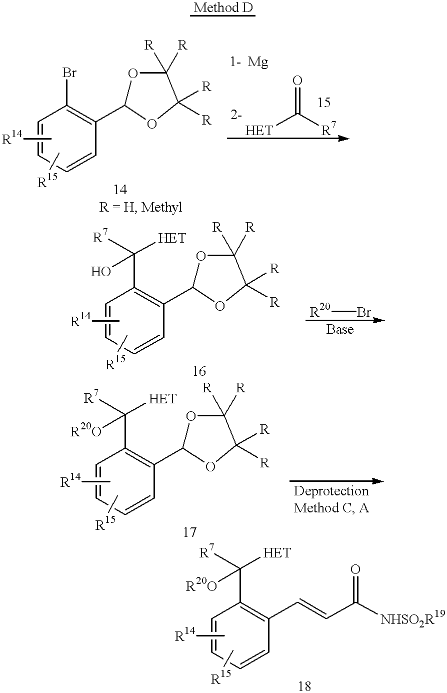

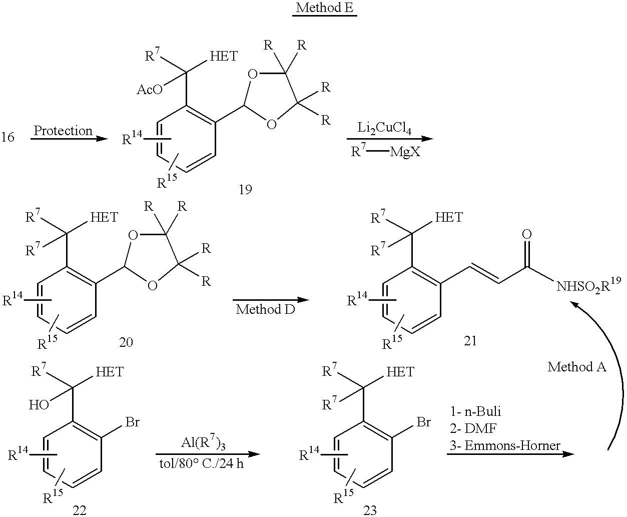

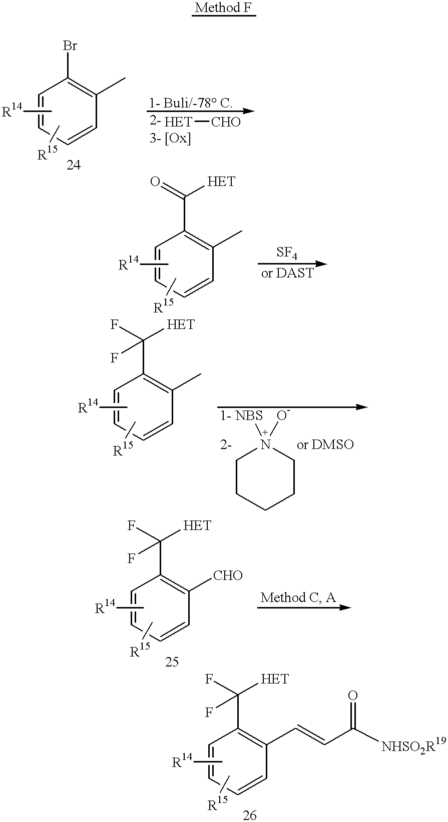

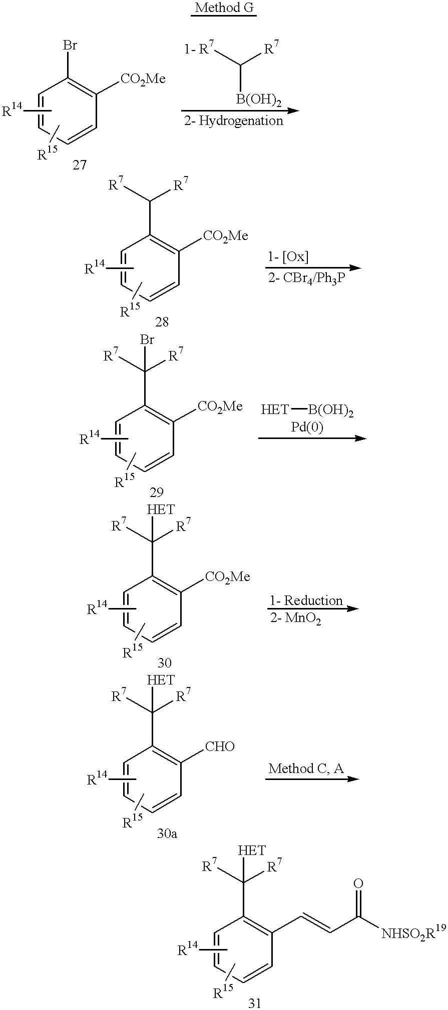

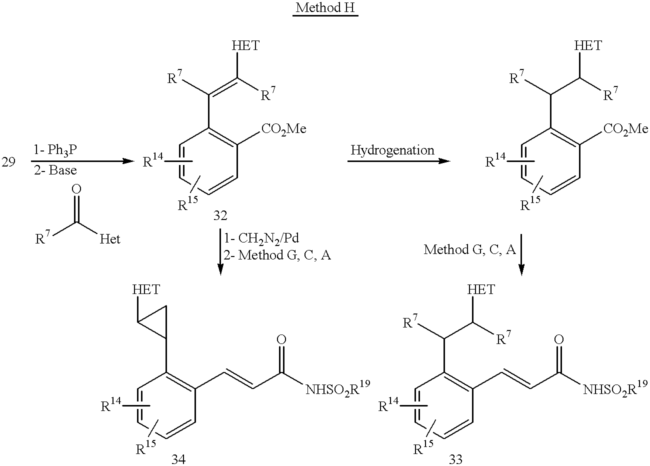

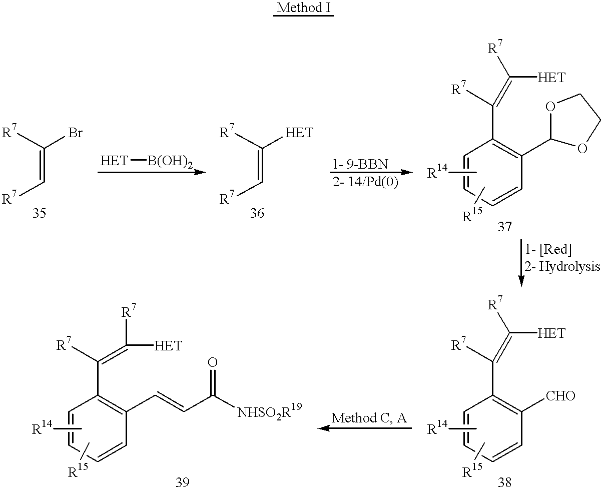

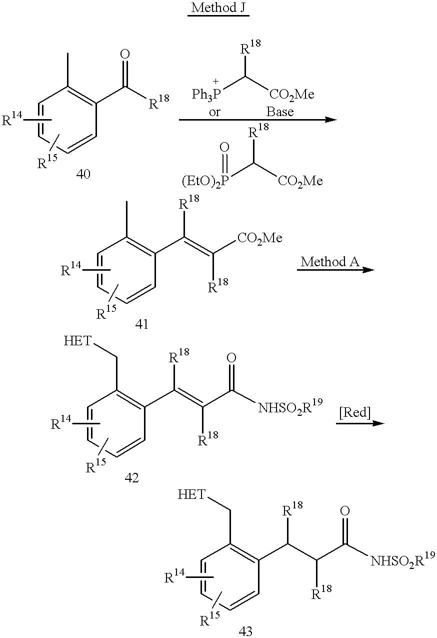

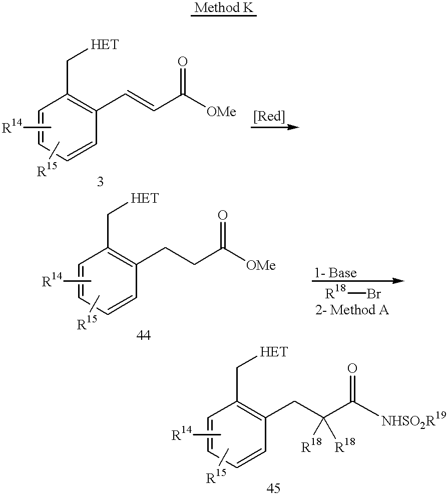

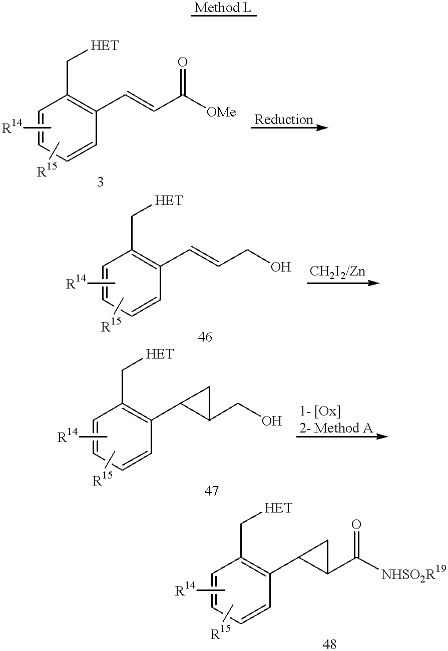

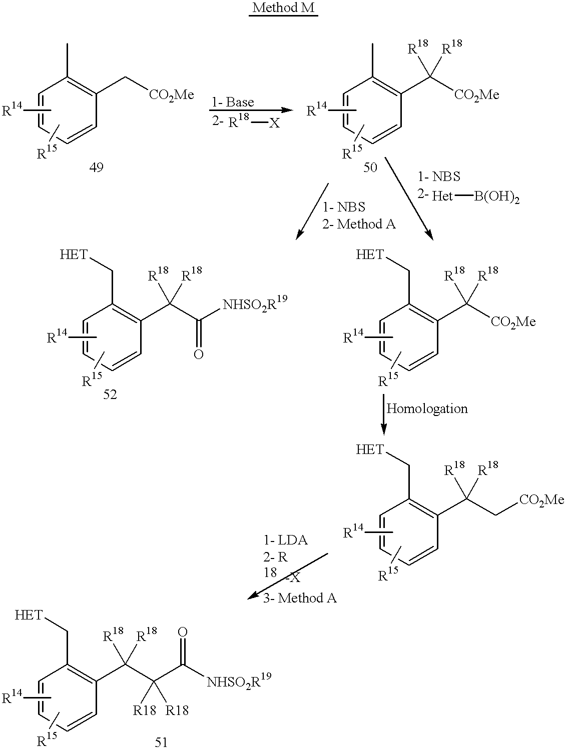

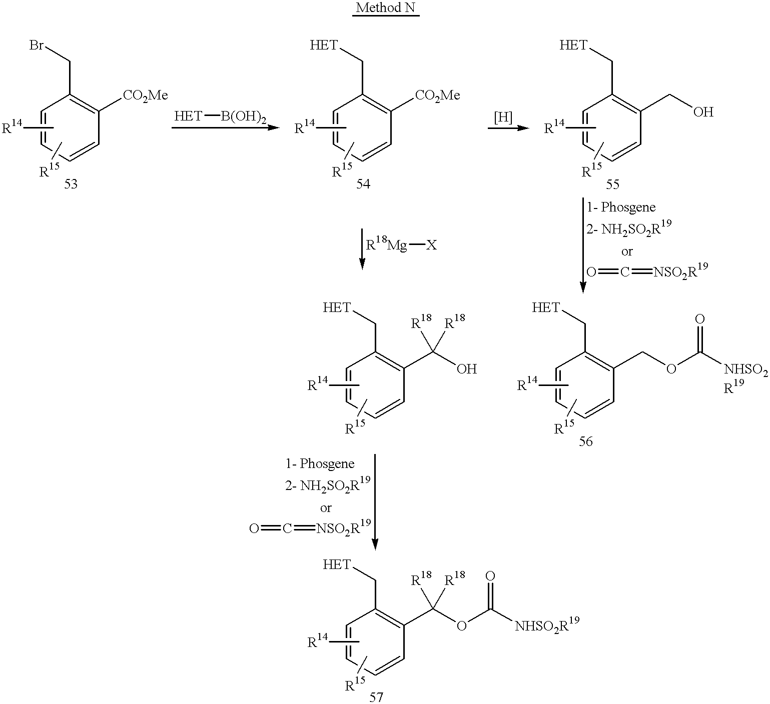

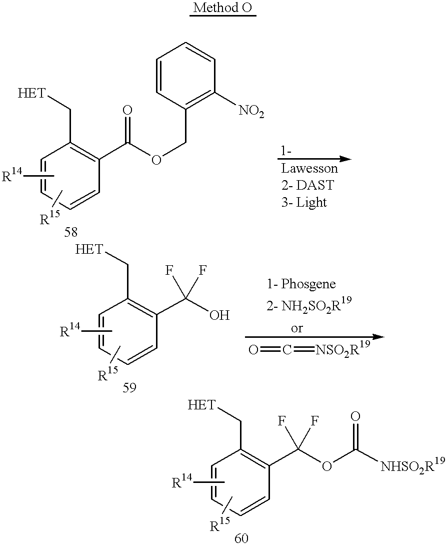

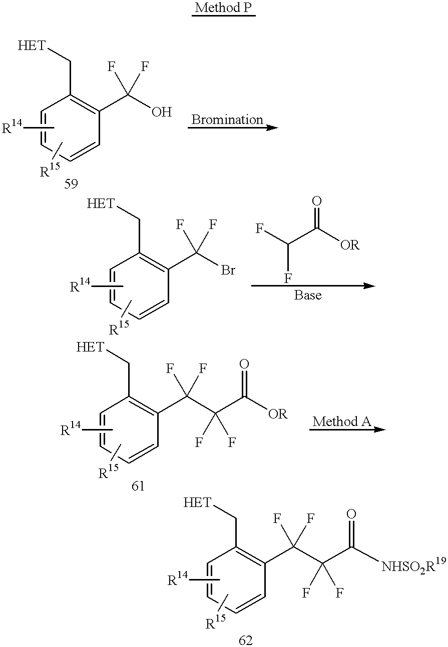

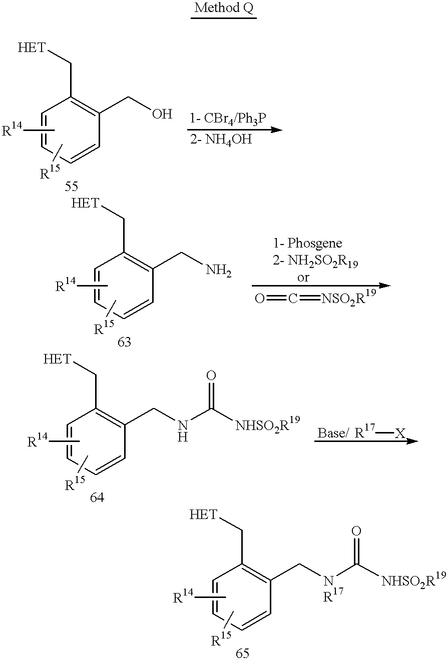

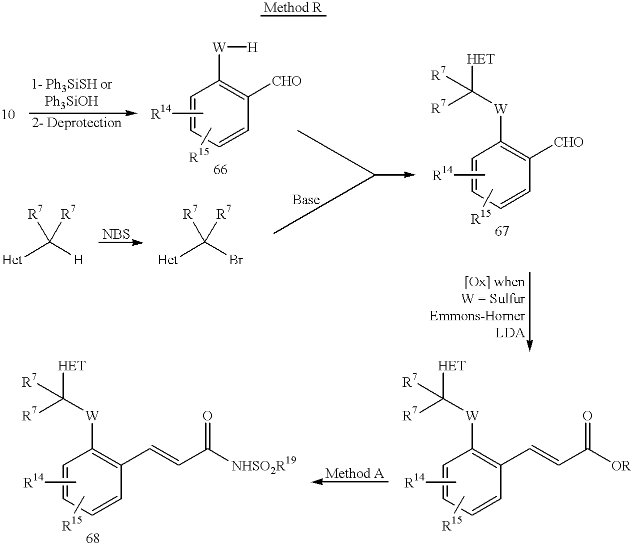

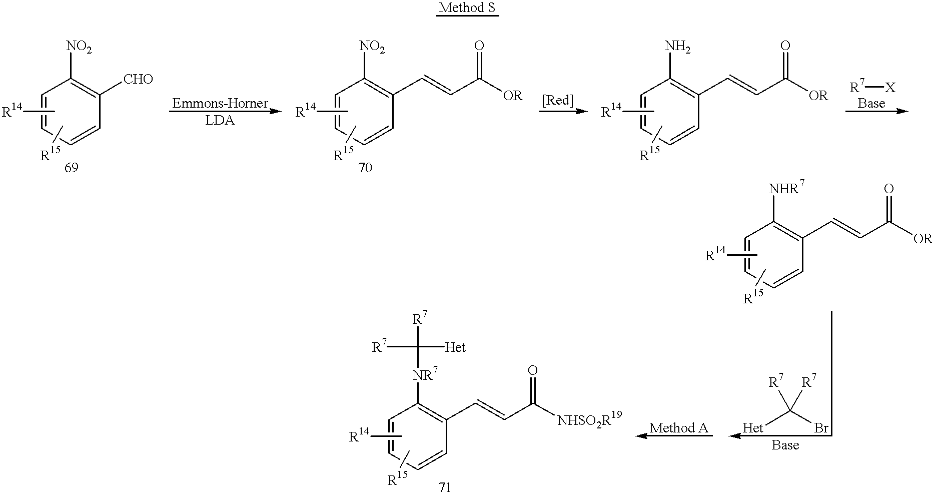

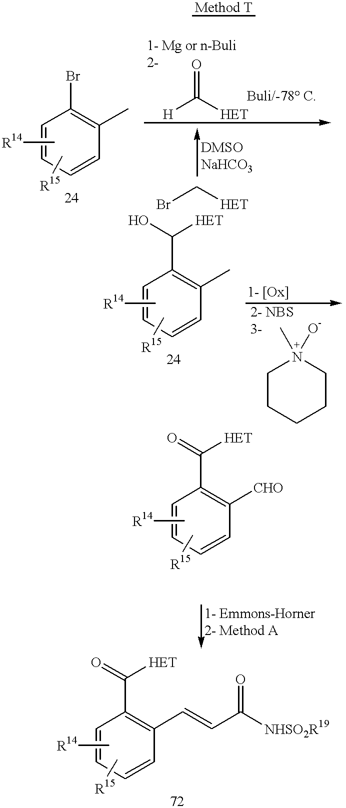

Examples of compounds which can be synthesized as described above are shown in Tables I and II below.

TABLE I

| | |
|---|---|
| | Ia |
| 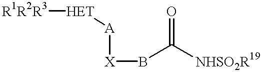 | |

| R¹R²R³-Het | A | X | B | R¹⁹ | Cpd |
|---|---|---|---|---|---|
| 1-naphthyl | CH₂ | 1,2-Ph | CH═CH | Ph(F)₅ | 1 |
| 2-naphthyl | S(O)₂ | 1,2-Ph | CH═CH | Ph(F)₅ | 2 |
| 1-(3-Me)- | CH₂ | 1,2-Ph | CH═CH | 2-thienyl | 3 |
| indolyl | | | | | |
| 2-naplithyl | CH₂ | 1,2-Ph | CH═CH | 2-thienyl | 4 |
| 2-naphthyl | S(O)₂ | 1,2-Ph | CH═CH | Ph | 5 |
| 1-(3-Me)- | S(O)₂ | 1,2-Ph | CH═CH | 2-thienyl | 6 |
| indolyl | | | | | |
| 2-naphthyl | S(O)₂ | 1,2-Ph | CH═CH | 3,5-di-(CF₃)-Ph | 7 |
| 3,4-di-Cl-Ph | CH₂ | 1,2-Ph | CH═CH | 2-thienyl | 8 |
| 2-naphthyl | S(O)₂ | 1,2-Ph | CH═CH | 2-thienyl | 9 |
| 2,4-di-Cl-Ph | CH₂ | 1,2-Ph | CH═CH | 2-thienyl | 10 |
| 1-naphthyl | S(O)₂ | 1,2-Ph | CH═CH | Ph(F)5 | 11 |
| 1-naphthyl | S(O)₂ | 1,2-Ph | CH═CH | 3,5-di-(CF₃)-Ph | 12 |
| 2-naphthyl | S(O)₂ | 1,2-Ph | 1,2-c-Pr | 2-thienyl | 13 |
| 3-Cl-4-F-Ph | CH₂ | 1,2-Ph | CH═CH | 2-thienyl | 14 |
| 1-naphthyl | CH₂ | 1,2-Ph | CH═CH | 2-thienyl | 15 |
| 3,4-di-Cl-Ph | S(O)₂ | 1,2-Ph | CH═CH | 2-thienyl | 16 |
| 4-MeS-Ph | CH₂ | 1,2-Ph | CH═CH | 2-thienyl | 17 |
| 4-Cl-Ph | CH₂ | 1,2-Ph | CH═CH | 2-thienyl | 18 |
| 2-naphthyl | S | 1,2-Ph | CH═CH | 2-thienyl | 19 |
| 2-naphthyl | O—CH₂ | 1,2-Ph | CH═CH | 2-thienyl | 20 |
| 2-naphthyl | S(O) | 1,2-Ph | CH═CH | 2-thienyl | 21 |
| 1-naphthyl | S(O)₂ | 1,2-Ph | CH═CH | Ph | 22 |
| 2-benzo- | CH₂ | 1,2-Ph | CH═CH | 2-thienyl | 23 |
| furanyl | | | | | |
| 3,5-di-Cl-Ph | CH₂ | 1,2-Ph | CH═CH | 2-thienyl | 24 |
| 1-naphthyl | S(O)₂ | 1,2-Ph | CH═CH | 3,5-di-(CF₃)-Ph | 25 |
| 1-naphthyl | S(O)₂ | 1,2-Ph | CH═CH | 2-thienyl | 26 |
| 1,3-benzodi- | CH₂ | 1,2-Ph | CH═CH | 2-thienyl | 27 |
| oxol-4-yl | | | | | |
| 2-naphthyl | O | 1,2-Ph | CH═CH | 2-thienyl | 28 |
| (2-Bn—C) | CH₂ | 1,2-Ph | CH₂—O | 2-thienyl | 29 |
| (2-Bn—C) | CH₂ | 1,2-Ph | CH₂CH₂ | 2-thienyl | 30 |
| 2-naphthyl | S(O)₂ | 1,2-Ph | CH₂—O | 2-thienyl | 31 |
| 3-(Qn)-Ph | CH₂ | 1,2-Ph | CH₂—O | 2-thienyl | 32 |
| 2-(6-Bn—O—) | CH₂ | 1,2-Ph | CH═CH | 2-thienyl | 33 |
| naphthyl | | | | | |
| 3-(Qn)-Ph | SO | 1,2-Ph | CH₂—O | 2-thienyl | 34 |
| 3-(Qn)-Ph | CHOH | 1,2-Ph | CH₂—O | 2-thienyl | 35 |
| 3-(Qn)-Ph | S(O)₂ | 1,2-Ph | CH₂—O | Ph | 36 |
| 3-(Qn)-Ph | O—CH₂ | 1,2-Ph | CH₂—O | 2-thienyl | 37 |
| 3-((3-tol- | O—CH₂ | 1,2-Ph | CH₂—O | 2-thienyl | 38 |
| yl)D)-Ph | | | | | |
| 3-(Qn)-Ph | C(OH)Me | 1,2-Ph | CH₂—O | Ph | 39 |
| 3-(Qn)-Ph | S | 1,2-Ph | CH₂—O | 2-thienyl | 40 |
| 3-(Qn)-Ph | O | 1,2-Ph | CH₂—O | Ph | 41 |
| 3-(Qn)-Ph | C═O | 1,2-Ph | CH₂—O | 2-thienyl | 42 |
| 3-(Qn)-Ph | O | 1,2-Ph | C(CH₃)₂—OH | 2-thienyl | 43 |
| | | | -OH | | |
| 3-(Qn)-Ph | O | 1,2-Ph | CH₂-O | 2-thienyl | 44 |
| 2-naphthyl | CH₂ | 1,2-Ph | 1,2-c-Pr | 2-thienyl | 45 |
| 2-(6-Bn—O—) | CH₂ | 1,2-Ph | CH═CH | 2-MeO-5- | 46 |
| naphthyl | | | | Br-Ph | |
| 2-naphthyl | CH₂ | 1,2-Ph | 1,2-c-Pr | 3,4-di-Cl-Ph | 47 |
| 2-naphthyl | OH₂ | 1,2-Ph | 1,2-c-Pr | 4-F-Ph | 48 |
| 2-naphthyl | CH₂ | 1,2-Ph | 1,2-c-Pr | 4-Cl-Ph | 49 |
| 2-naphthyl | CH₂ | 1,2-Ph | 1,2-c-Pr | 4-n-Pr-Ph | 50 |
| 2-naphthyl | CH₂ | 1,2-Ph | 1,2-c-Pr | 2,5-di-Ci-thienyl | 51 |
| 2-naphthyl | CH₂ | 1,2-Ph | 1,2-c-Pr | 2-Ph-ethenyl | 52 |
| 2-naphthyl | CH₂ | 1,2-Ph | 1,2-c-Pr | 3-Cl-4-F-Ph | 53 |
| 2-naphthyl | CH₂ | 1,2-Ph | 1,2-c-Pr | 4-MeO-Ph | 54 |
| 2-naphthyl | CH₂ | 1,2-Ph | 1,2-c-Pr | 3-Br-Ph | 55 |
| 2-naphthyl | CH₂ | 1,2-Ph | 1,2-c-Pr | 2,5-di-Me-Ph | 56 |
| 2-naphthyl | CH₂ | 1,2-Ph | 1,2-c-Pr | 2-NO₂-4-Cl-Ph | 57 |
| 2-naphthyl | CH₂ | 1,2-Ph | 1,2-c-Pr | 2-MeOC(O)-Ph | 58 |
| 2-naphthyl | CH₂ | 1,2-Ph | 1,2-c-Pr | 2,4-di-F-Ph | 59 |
| 2-naphthyl | CH₂ | 1,2-Ph | 1,2-c-Pr | 4-n-butyl-Ph | 60 |
| 2-naphthyl | CH₂ | 1,2-Ph | 1,2-c-Pr | n-butyl | 61 |
| 2-naphthyl | CH₂ | 1,2-Ph | 1,2-c-Pr | 2,5-di-MeO-Ph | 62 |
| 2-naphthyl | CH₂ | 1,2-Ph | 1,2-c-Pr | 3-CF₃-Ph | 63 |
| 2-naphthyl | CH₂ | 1,2-Ph | 1,2-c-Pr | 3,5-di-F-Ph | 64 |
| 2-naphthyl | CH₂ | 1,2-Ph | 1,2-c-Pr | 3,5-di-Cl-Ph | 65 |
| 2-naphthyl | CH₂ | 1,2-Ph | 1,2-c-Pr | 4-(1-OH-1-Me)- | 66 |
| | | | | Et-Ph | |
| 2-naphthyl | CH₂ | 1,2-Ph | 1,2-c-Pr | 4-(HO-Me)-Ph | 67 |
| 2-naphthyl | CH₂ | 1,2-Ph | 1,2-c-Pr | 3-(HO-Me)-Ph | 68 |
| 2-naphthyl | CH₂ | 1,2-Ph | 1,2-c-Pr | 4-(MeSO₂)-Ph | 69 |
| 2-naphthyl | CH₂ | 1,2-Ph | 1,2-c-Pr | 3-(MeSO₂)-Ph | 70 |
| 2-naphthyl | CH₂ | 1,2-Ph | 1,2-c-Pr | 4-(n-Pr-SO₂)-Ph | 71 |
| 2-naphthyl | CH₂ | 1,2-Ph | 1,2-c-Pr | 4-((bis-CF₃)- | 72 |
| | | | | HO-methyl)-Ph | |
| 2-naphthyl | CH₂ | 1,2-Ph | 1,2-c-Pr | 4-(Bn-O)-Ph | 73 |
| 2-naphthyl | CH₂ | 1,2-Ph | 1,2-c-Pr | 4-(1-MeO-meth- | 74 |
| | | | | yl-1-Me)-Ph | |
| 2-naphthyl | CH₂ | 1,2-Ph | 1,2-c-Pr | 4-Me2N-Ph | 75 |
| 2-naphthyl | CH₂ | 1,2-Ph | 1,2-c-Pr | c-Hex | 76 |
| 2-naphthyl | CH₂ | 1,2-Ph | 1,2-c-Pr | c-Pen | 77 |
| 2-naphthyl | CH₂ | 1,2-Ph | 1,2-c-Pr | 2-oxazolyl | 78 |
| 2-naphthyl | CH₂ | 1,2-Ph | 1,2-c-Pr | 2-naphthyl | 79 |
| 2-naphthyl | CH₂ | 1,2-Ph | 1,2-c-Pr | 1-thiazolyl | 80 |
| 2-naphthyl | CH₂ | 1,2-Ph | 1,2-c-Pr | 1-imidazolyl | 81 |
| 2-naphthyl | CH₂ | 1,2-Ph | 1,2-c-Pr | 2-furanyl | 82 |
| 2-naphthyl | CH₂ | 1,2-Ph | 1,2-c-Pr | 3-(2-Cl)furanyl | 83 |
| 2-naphthyl | CH₂ | 1,2-Ph | 1,2-c-Pr | 2-pyridinyl | 84 |
| 2-naphthyl | CH₂ | 1,2-Ph | 1,2-c-Pr | 2-(4-Cl)pyridinyl | 85 |
| 2-naphthyl | CH₂ | 1,2-Ph | 1,2-c-Pr | 3-indolyl | 86 |
| 2-naphthyl | CH₂ | 1,2-Ph | 1,2-c-Pr | 4-NO₂-Ph | 87 |
| 2-naphthyl | CH₂ | 1,2-Ph | 1,2-c-Pr | 4-CN-Ph | 88 |
| 2-naphthyl | S(O)₂ | 1,2-Ph | 1,2-c-Pr | 4-(1-OH-1-Me)- | 89 |
| | | | | ethyl-Ph | |
| 2-naphthyl | S(O)₂ | 1,2-Ph | 1,2-c-Pr | 4-(HO-Me)-Ph | 90 |
| 2-naphthyl | S(O)₂ | 1,2-Ph | 1,2-c-Pr | 3-(HO-Me)-Ph | 91 |
| 2-naphthyl | S(O)₂ | 1,2-Ph | 1,2-c-Pr | 2,5-di-Me-Ph | 92 |
| 2-naphthyl | S(O)₂ | 1,2-Ph | 1,2-c-Pr | 2-MeOC(O)-Ph | 93 |
| 2-naphthyl | S(O)₂ | 1,2-Ph | 1,2-c-Pr | 2,4-di-F-Ph | 94 |
| 2-naphthyl | S(O)₂ | 1,2-Ph | 1,2-c-Pr | 4-(Me-SO₂)-Ph | 95 |
| 2-naphthyl | S(O)₂ | 1,2-Ph | 1,2-c-Pr | 3-(Me-SO₂)-Ph | 96 |
| 2-naphthyl | S(O)₂ | 1,2-Ph | 1,2-c-Pr | 4-(n-Pr-SO₂)-Ph | 97 |
| 2-naphthyl | S(O)₂ | 1,2-Ph | 1,2-c-Pr | 4-n-butyl-Ph | 98 |
| 2-naphthyl | S(O)₂ | 1,2-Ph | 1,2-c-Pr | 3,5-(di-CF₃)-Ph | 99 |
| 2-naphthyl | S(O)₂ | 1,2-Ph | 1,2-c-Pr | 4-((bis-CF₃)- | 100 |
| | | | | HO-Me)-Ph | |
| 2-naphthyl | S(O)₂ | 1,2-Ph | 1,2-c-Pr | 3-Br-Ph | 101 |
| 2-naphthyl | S(O)₂ | 1,2-Ph | 1,2-c-Pr | 4-(Bn-O)-Ph | 102 |
| 2-naphthyl | S(O)₂ | 1,2-Ph | 1,2-c-Pr | 2-NO₂-4-Cl-Ph | 103 |
| 2-naphthyl | S(O)₂ | 1,2-Ph | 1,2-c-Pr | 4-i-Pr-Ph | 104 |
| 2-naphthyl | S(O)₂ | 1,2-Ph | 1,2-c-Pr | 4-(1-MeO- | 105 |
| | | | | methyl-1-Me)-Ph | |
| 2-naphthyl | S(O)₂ | 1,2-Ph | 1,2-c-Pr | 4-Me-O-Ph | 106 |
| 2-naphthyl | S(O)₂ | 1,2-Ph | 1,2-c-Pr | 4-Me₂N-Ph | 107 |
| 2-naphthyl | S(O)₂ | 1,2-Ph | 1,2-c-Pr | 3,4-di-Cl-Ph | 108 |
| 2-naphthyl | S(O)₂ | 1,2-Ph | 1,2-c-Pr | 3,4-di-F-Ph | 109 |
| 2-naphthyl | S(O)₂ | 1,2-Ph | 1,2-c-Pr | 4-F-Ph | 110 |
| 2-naphthyl | S(O)₂ | 1,2-Ph | 1,2-c-Pr | c-Hex | 111 |
| 2-naphthyl | S(O)₂ | 1,2-Ph | 1,2-c-Pr | c-Pen | 112 |
| 2-naphthyl | S(O)₂ | 1,2-Ph | 1,2-c-Pr | 4-oxazolyl | 113 |
| 2-naphthyl | S(O)₂ | 1,2-Ph | 1,2-c-Pr | n-butyl | 114 |
| 2-naphthyl | S(O)₂ | 1,2-Ph | 1,2-c-Pr | 4-Cl-Ph | 115 |
| 2-naphthyl | S(O)₂ | 1,2-Ph | 1,2-c-Pr | 4-n-Pr-Ph | 116 |
| 2-naphthyl | S(O)₂ | 1,2-Ph | 1,2-c-Pr | 2-naphthyl | 117 |
| 2-naphthyl | S(O)₂ | 1,2-Ph | 1,2-c-Pr | 1-thiazoyl | 118 |
| 2-naphthyl | S(O)₂ | 1,2-Ph | 1,2-c-Pr | 1-imidazoyl | 119 |
| 2-naphthyl | S(O)₂ | 1,2-Ph | 1,2-c-Pr | 2,5-di-Meo-Ph | 120 |
| 2-naphthyl | S(O)₂ | 1,2-Ph | 1,2-c-Pr | 3-CF₃-Ph | 121 |
| 2-naphthyl | S(O)₂ | 1,2-Ph | 1,2-c-Pr | 2,5-di-Cl-thienyl | 122 |
| 2-naphthyl | S(O)₂ | 1,2-Ph | 1,2-c-Pr | 2-furanyl | 123 |
| 2-naphthyl | S(O)₂ | 1,2-Ph | 1,2-c-Pr | 3-(2-Cl)furanyl | 124 |
| 2-naphthyl | S(O)₂ | 1,2-Ph | 1,2-c-Pr | 2-pyridinyl | 125 |
| 2-naphthyl | S(O)₂ | 1,2-Ph | 1,2-c-Pr | 2-Ph-ethenyl | 126 |
| 2-naphthyl | S(O)₂ | 1,2-Ph | 1,2-c-Pr | 3,5-di-F-Ph | 127 |
| 2-naphthyl | S(O)₂ | 1,2-Ph | 1,2-c-Pr | 3,5-di-Cl-Ph | 128 |
| 2-naphthyl | S(O)₂ | 1,2-Ph | 1,2-c-Pr | 2-(4-Cl)pyridinyl | 129 |
| 2-naphthyl | S(O)₂ | 1,2-Ph | 1,2-c-Pr | 3-indolyl | 130 |
| 2-naphthyl | S(O)₂ | 1,2-Ph | 1,2-c-Pr | 4-NO₂-Ph | 131 |
| 2-naphthyl | S(O)₂ | 1,2-Ph | 1,2-c-Pr | 4-CN-Ph | 132 |
| 2-naphthyl | S(O)₂ | 1,2-Ph | 1,2-c-Pr | 3-Cl-4-F-Ph | 133 |
| 1-(3-Me)- | CH₂ | 1,2-Ph | 1,2-c-Pr | 3,5-(di-CF₃)-Ph | 134 |
| indolyl | | | | | |
| 1-(3-Me)- | CH₂ | 1,2-Ph | 1,2-c-Pr | 4-i-Pr-Ph | 135 |
| indolyl | | | | | |
| 1-(3-Me)- | CH₂ | 1,2-Ph | 1,2-c-Pr | 3,4-di-Cl-Ph | 136 |
| indolyl | | | | | |
| 1-(3-Me)- | CH₂ | 1,2-Ph | 1,2-c-Pr | 3,4-di-F-Ph | 137 |
| indolyl | | | | | |
| 1-(3-Me)- | CH₂ | 1,2-Ph | 1,2-c-Pr | 4-F-Ph | 138 |
| indolyl | | | | | |
| 1-(3-Me)- | CH₂ | 1,2-Ph | 1,2-c-Pr | 4-Cl-Ph | 139 |
| indolyl | | | | | |
| 1-(3-Me)- | CH₂ | 1,2-Ph | 1,2-c-Pr | 4-n-Pr-Ph | 140 |
| indolyl | | | | | |
| 1-(3-Me)- | CH₂ | 1,2-Ph | 1,2-c-Pr | 2,5-di-Cl-thien- | 141 |
| indolyl | | | | 3-yl | |
| 1-(3-Me)- | CH₂ | 1,2-Ph | 1,2-c-Pr | 2-Ph-ethenyl | 142 |
| indolyl | | | | | |
| 1-(3-Me)- | CH₂ | 1,2-Ph | 1,2-c-Pr | 3-Cl-4-F-Ph | 143 |
| indolyl | | | | | |
| 1-(3-Me)- | CH₂ | 1,2-Ph | 1,2-c-Pr | 4-MeO-Ph | 144 |
| indolyl | | | | | |
| 1-(3-Me)- | CH₂ | 1,2-Ph | 1,2-c-Pr | 3-Br-Ph | 145 |
| indolyl | | | | | |
| 1-(3-Me)- | CH₂ | 1,2-Ph | 1,2-c-Pr | 2,5-di-Me-Ph | 146 |
| indolyl | | | | | |
| 1-(3-Me)- | CH₂ | 1,2-Ph | 1,2-c-Pr | 2-NO₂-4-Cl-Ph | 147 |
| indolyl | | | | | |
| 1-(3-Me)- | CH₂ | 1,2-Ph | 1,2-c-Pr | 2-MeOC(O)-Ph | 148 |
| indolyl | | | | | |
| 1-(3-Me)- | CH₂ | 1,2-Ph | 1,2-c-Pr | 2,4-di-F-Ph | 149 |
| indolyl | | | | | |
| 1-(3-Me)- | CH₂ | 1,2-Ph | 1,2-c-Pr | 4-n-butyl-Ph | 150 |
| indolyl | | | | | |
| 1-(3-Me)- | CH₂ | 1,2-Ph | 1,2-c-Pr | n-butyl | 151 |
| indolyl | | | | | |
| 1-(3-Me)- | CH₂ | 1,2-Ph | 1,2-c-Pr | 2,5-di-MeO-Ph | 152 |
| indolyl | | | | | |
| 1-(3-Me)- | CH₂ | 1,2-Ph | 1,2-c-Pr | 3-CF₃-Ph | 153 |
| indolyl | | | | | |
| 1-(3-Me)- | CH₂ | 1,2-Ph | 1,2-c-Pr | 3,5-di-F-Ph | 154 |
| indolyl | | | | | |
| 1-(3-Me)- | CH₂ | 1,2-Ph | 1,2-c-Pr | 3,5-di-Cl-Ph | 155 |
| indolyl | | | | | |
| 1-(3-Me)- | CH₂ | 1,2-Ph | 1,2-c-Pr | 4-(1-OH-1-Me)- | 156 |
| indolyl | | | | ethyl-Ph | |
| 1-(3-Me)- | CH₂ | 1,2-Ph | 1,2-c-Pr | 4-(HO-Me)-Ph | 157 |
| indolyl | | | | | |
| 1-(3-Me)- | CH₂ | 1,2-Ph | 1,2-c-Pr | 3-(HO-Me)-Ph | 158 |
| indolyl | | | | | |
| 1-(3-Me)- | CH₂ | 1,2-Ph | 1,2-c-Pr | 4-(Me-SO₂)-Ph | 159 |
| indolyl | | | | | |
| 1-(3-Me)- | CH₂ | 1,2-Ph | 1,2-c-Pr | 3-(Me-SO₂)-Ph | 160 |
| indolyl | | | | | |
| 1-(3-Me)- | CH₂ | 1,2-Ph | 1,2-c-Pr | 4-(n-Pr-SO₂)-Ph | 161 |
| indolyl | | | | | |
| 1-(3-Me)- | CH₂ | 1,2-Ph | 1,2-c-Pr | 4-((bis-CF₃)- | 162 |
| indolyl | | | | HO-methyl)-Ph | |
| 1-(3-Me)- | CH₂ | 1,2-Ph | 1,2-c-Pr | 4-(Bn-O)-Ph | 163 |
| indolyl | | | | | |
| 1-(3-Me)- | CH₂ | 1,2-Ph | 1,2-c-Pr | 4-(1-MeO-1-Me)- | 164 |
| indolyl | | | | ethyl-Ph | |
| 1-(3-Me)- | CH₂ | 1,2-Ph | 1,2-c-Pr | 4-Me₂N-Ph | 165 |
| indolyl | | | | | |
| 1-(3-Me)- | CH₂ | 1,2-Ph | 1,2-c-Pr | c-Hex | 166 |
| indolyl | | | | | |
| 1-(3-Me)- | CH₂ | 1,2-Ph | 1,2-c-Pr | c-Pen | 167 |
| indolyl | | | | | |
| 1-(3-Me)- | CH₂ | 1,2-Ph | 1,2-c-Pr | 1-morpholinyl | 168 |
| indolyl | | | | | |
| 1-(3-Me)- | CH₂ | 1,2-Ph | 1,2-c-Pr | 2-naphthyl | 169 |
| indolyl | | | | | |
| 1-(3-Me)- | CH₂ | 1,2-Ph | 1,2-c-Pr | 1-thiazolyl | 170 |
| indolyl | | | | | |
| 1-(3-Me)- | CH₂ | 1,2-Ph | 1,2-c-Pr | 1-imidazolyl | 171 |
| indolyl | | | | | |
| 1-(3-Me)- | CH₂ | 1,2-Ph | 1,2-c-Pr | 3-furanyl | 172 |
| indolyl | | | | | |
| 1-(3-Me)- | CH₂ | 1,2-Ph | 1,2-c-Pr | 4-(2-Cl)furanyl | 173 |
| indolyl | | | | | |
| 1-(3-Me)- | CH₂ | 1,2-Ph | 1,2-c-Pr | 2-pyridinyl | 174 |
| indolyl | | | | | |
| 1-(3-Me)- | CH₂ | 1,2-Ph | 1,2-c-Pr | 2-(4-Cl)pyridinyl | 175 |
| indolyl | | | | | |
| 1-(3-Me)- | CH₂ | 1,2-Ph | 1,2-c-Pr | 3-indolyl | 176 |
| indolyl | | | | | |
| 1-(3-Me)- | CH₂ | 1,2-Ph | 1,2-c-Pr | 4-NO₂-Ph | 177 |
| indolyl | | | | | |
| 1-(3-Me)- | CH₂ | 1,2-Ph | 1,2-c-Pr | 4-CN-Ph | 178 |
| indolyl | | | | | |
| 1-(3-Me)- | SO₂ | 1,2-Ph | 1,2-c-Pr | 3,5-(di-CF₃)-Ph | 179 |
| indolyl | | | | | |
| 1-(3-Me)- | SO₂ | 1,2-Ph | 1,2-c-Pr | 4-i-Pr-Ph | 180 |
| indolyl | | | | | |
| 1-(3-Me)- | SO₂ | 1,2-Ph | 1,2-c-Pr | 3,4-di-Cl-Ph | 181 |
| indolyl | | | | | |
| 1-(3-Me)- | SO₂ | 1,2-Ph | 1,2-c-Pr | 3,4-di-F-Ph | 182 |
| indolyl | | | | | |
| 1-(3-Me)- | SO₂ | 1,2-Ph | 1,2-c-Pr | 4-F-Ph | 183 |
| indolyl | | | | | |
| 1-(3-Me)- | SO₂ | 1,2-Ph | 1,2-c-Pr | 4-Cl-Ph | 184 |
| indolyl | | | | | |
| 1-(3-Me)- | SO₂ | 1,2-Ph | 1,2-c-Pr | 4-n-Pr-Ph | 185 |
| indolyl | | | | | |
| 1-(3-Me)- | SO₂ | 1,2-Ph | 1,2-c-Pr | 2,5-di-Ci-thienyl | 186 |
| indolyl | | | | | |
| 1-(3-Me)- | SO₂ | 1,2-Ph | 1,2-c-Pr | 2-Ph-ethenyl | 187 |
| indolyl | | | | | |
| 1-(3-Me)- | SO₂ | 1,2-Ph | 1,2-c-Pr | 3-C1-4-F-Ph | 188 |
| indolyl | | | | | |
| 1-(3-Me)- | SO₂ | 1,2-Ph | 1,2-c-Pr | 4-MeO-Ph | 189 |
| indolyl | | | | | |
| 1-(3-Me)- | SO₂ | 1,2-Ph | 1,2-c-Pr | 3-Br-Ph | 190 |
| indolyl | | | | | |
| 1-(3-Me)- | SO₂ | 1,2-Ph | 1,2-c-Pr | 2,5-di-Me-Ph | 191 |
| indolyl | | | | | |
| 1-(3-Me)- | SO₂ | 1,2-Ph | 1,2-c-Pr | 2-NO₂-4-Cl-Ph | 192 |
| indolyl | | | | | |
| 1-(3-Me)- | SO₂ | 1,2-Ph | 1,2-c-Pr | 2-MeOC(O)-Ph | 193 |
| indolyl | | | | | |
| 1-(3-Me)- | SO₂ | 1,2-Ph | 1,2-c-Pr | 2,4-di-F-Ph | 194 |
| indolyl | | | | | |
| 1-(3-Me)- | SO₂ | 1,2-Ph | 1,2-c-Pr | 4-n-butyl-Ph | 195 |
| indolyl | | | | | |
| 1-(3-Me)- | SO₂ | 1,2-Ph | 1,2-c-Pr | n-butyl | 196 |
| indolyl | | | | | |
| 1-(3-Me)- | SO₂ | 1,2-Ph | 1,2-c-Pr | 2,5-di-MeO-Ph | 197 |
| indolyl | | | | | |
| 1-(3-Me)- | SO₂ | 1,2-Ph | 1,2-c-Pr | 3-CF₃-Ph | 198 |
| indolyl | | | | | |
| 1-(3-Me)- | SO₂ | 1,2-Ph | 1,2-c-Pr | 3,5-di-F-Ph | 199 |
| indolyl | | | | | |
| 1-(3-Me)- | SO₂ | 1,2-Ph | 1,2-c-Pr | 3,5-di-Cl-Ph | 200 |
| indolyl | | | | | |
| 1-(3-Me)- | SO₂ | 1,2-Ph | 1,2-c-Pr | 4-(1-OH-1-Me)- | 201 |
| indolyl | | | | ethyl-Ph | |
| 1-(3-Me)- | SO₂ | 1,2-Ph | 1,2-c-Pr | 4-(HO-Me)-Ph | 202 |
| indolyl | | | | | |
| 1-(3-Me)- | SO₂ | 1,2-Ph | 1,2-c-Pr | 3-(HO-Me)-Ph | 203 |
| indolyl | | | | | |
| 1-(3-Me)- | SO₂ | 1,2-Ph | 1,2-c-Pr | 4-(Me-SO₂)-Ph | 204 |
| indolyl | | | | | |
| 1-(3-Me)- | SO₂ | 1,2-Ph | 1,2-c-Pr | 3-(Me-SO₂)-Ph | 205 |
| indolyl | | | | | |
| 1-(3-Me)- | SO₂ | 1,2-Ph | 1,2-c-Pr | 4-(n-Pr-SO₂)-Ph | 206 |
| indolyl | | | | | |
| 1-(3-Me)- | SO₂ | 1,2-Ph | 1,2-c-Pr | 4-((bis-CF₃)- | 207 |
| indolyl | | | | HO-Me)-Ph | |
| 1-(3-Me)- | SO₂ | 1,2-Ph | 1,2-c-Pr | 4-(Bn-O)-Ph | 208 |
| indolyl | | | | | |
| 1-(3-Me)- | SO₂ | 1,2-Ph | 1,2-c-Pr | 4-((1-MeOCH₂- | 209 |
| indolyl | | | | 1-Me-1-ethyl-)-Ph | |
| 1-(3-Me)- | SO₂ | 1,2-Ph | 1,2-c-Pr | 4-Me₂N-Ph | 210 |
| indolyl | | | | | |
| 1-(3-Me)- | SO₂ | 1,2-Ph | 1,2-c-Pr | c-Hex | 211 |
| indolyl | | | | | |
| 1-(3-Me)- | SO₂ | 1,2-Ph | 1,2-c-Pr | c-Pen | 212 |
| indolyl | | | | | |
| 1-(3-Me)- | SO₂ | 1,2-Ph | 1,2-c-Pr | 1-morpholinyl | 213 |
| indolyl | | | | | |
| 1-(3-Me)- | SO₂ | 1,2-Ph | 1,2-c-Pr | 2-naphthyl | 214 |
| indolyl | | | | | |
| 1-(3-Me)- | SO₂ | 1,2-Ph | 1,2-c-Pr | 2-thiazolyl | 215 |
| indolyl | | | | | |
| 1-(3-Me)- | SO₂ | 1,2-Ph | 1,2-c-Pr | 1-imidazolyl | 216 |
| indolyl | | | | | |
| 1-(3-Me)- | SO₂ | 1,2-Ph | 1,2-c-Pr | 2-furanyl | 217 |
| indolyl | | | | | |
| 1-(3-Me)- | SO₂ | 1,2-Ph | 1,2-c-Pr | 5-(2-Cl)furanyl | 218 |
| indolyl | | | | | |
| 1-(3-Me)- | SO₂ | 1,2-Ph | 1,2-c-Pr | 2-pyridinyl | 219 |
| indolyl | | | | | |
| 1-(3-Me)- | SO₂ | 1,2-Ph | 1,2-c-Pr | 2-(4-Cl)pyridinyl | 220 |
| indolyl | | | | | |
| 1-(3-Me)- | SO₂ | 1,2-Ph | 1,2-c-Pr | 3-indolyl | 221 |
| indolyl | | | | | |
| 1-(3-Me)- | SO₂ | 1,2-Ph | 1,2-c-Pr | 4-NO₂-Ph | 222 |
| indolyl | | | | | |
| 1-(3-Me)- | SO₂ | 1,2-Ph | 1,2-c-Pr | 4-CN-Ph | 223 |
| indolyl | | | | | |
| 2-naphthyl | CH₂ | 1,2-Ph | CH═CH | 3,5-(di-CF₃)-Ph | 224 |
| 2-naphthyl | CH₂ | 1,2-Ph | CH═CH | 4-i-Pr-Ph | 225 |
| 2-naphthyl | CH₂ | 1,2-Ph | CH═CH | 2,3-di-Cl-Ph | 226 |
| 2-naphthyl | CH₂ | 1,2-Ph | CH═CH | 3,4-di-F-Ph | 227 |
| 2-naphthyl | CH₂ | 1,2-Ph | CH═CH | 4-Cl-Ph | 228 |
| 2-naphthyl | CH₂ | 1,2-Ph | CH═CH | 4-F-Ph | 229 |
| 2-naphthyl | CH₂ | 1,2-Ph | CH═CH | 2,5-di-Ci-thienyl | 230 |
| 2-naphthyl | CH₂ | 1,2-Ph | CH═CH | 3-Cl-4-F-Ph | 231 |
| 2-naphthyl | CH₂ | 1,2-Ph | CH═CH | 4-MeO-Ph | 232 |
| 2-naphthyl | CH₂ | 1,2-Ph | CH═CH | butyl | 233 |
| 2-naphthyl | CH₂ | 1,2-Ph | CH═CH | 3-CF₃-Ph | 234 |
| 2-naphthyl | CH₂ | 1,2-Ph | CH═CH | 4-((1-OH-1-Me) | 235 |
| | | | | ethyl)-Ph | |
| 2-naphthyl | CH₂ | 1,2-Ph | CH═CH | 4-(Me-SO₂)-Ph | 236 |
| 2-naphthyl | CH₂ | 1,2-Ph | CH═CH | 4-(Bn-O)-Ph | 237 |
| 2-naphthyl | CH₂ | 1,2-Ph | CH═CH | c-Hex | 238 |
| 2-naphthyl | CH₂ | 1,2-Ph | CH═CH | 3-(1,2,5-thiadiazoyl) | 239 |
| 2-naphthyl | CH₂ | 1,2-Ph | CH═CH | 2-thiazolyl | 240 |
| 2-naphthyl | CH₂ | 1,2-Ph | CH═CH | 2-furanyl | 241 |
| 2-naphthyl | CH₂ | 1,2-Ph | CH═CH | 2-pyridinyl | 242 |
| 2-naphthyl | CH₂ | 1,2-Ph | CH═CH | 4-CN-Ph | 243 |
| 2-naphthyl | SO₂ | 1,2-Ph | CH═CH | 3,5-(di-CF₃)-Ph | 244 |
| 2-naphthyl | SO₂ | 1,2-Ph | CH═CH | 4-i-Pr-Ph | 245 |
| 2-naphthyl | SO₂ | 1,2-Ph | CH═CH | 2,3-di-Cl-Ph | 246 |
| 2-naphthyl | SO₂ | 1,2-Ph | CH═CH | 3,4-di-F-Ph | 247 |
| 2-naphthyl | SO₂ | 1,2-Ph | CH═CH | 4-Cl-Ph | 248 |
| 2-naphthyl | SO₂ | 1,2-Ph | CH═CH | 4-F-Ph | 249 |
| 2-naphthyl | SO₂ | 1,2-Ph | CH═CH | 2,5-di-Cl-thien-3-yl | 250 |
| 2-naphthyl | SO₂ | 1,2-Ph | CH═CH | 3-Cl-4-F-Ph | 251 |
| 2-naphthyl | SO₂ | 1,2-Ph | CH═CH | 4-MeO-Ph | 252 |
| 2-naphthyl | SO₂ | 1,2-Ph | CH═CH | butyl | 253 |
| 2-naphthyl | SO₂ | 1,2-Ph | CH═CH | 3-CF₃-Ph | 254 |
| 2-naphthyl | SO₂ | 1,2-Ph | CH═CH | 4-((1-OH-1-Me) | 255 |
| | | | | ethyl)-Ph | |
| 2-naphthyl | SO₂ | 1,2-Ph | CH═CH | 4-(Me-SO₂)-Ph | 256 |
| 2-naphthyl | SO₂ | 1,2-Ph | CH═CH | 4-(Bn—O)-Ph | 257 |
| 2-naphthyl | SO₂ | 1,2-Ph | CH═CH | c-Hex | 258 |
| 2-naphthyl | SO₂ | 1,2-Ph | CH═CH | 2-(1,3,4-thiadiazoyl) | 259 |
| 2-naphthyl | SO₂ | 1,2-Ph | CH═CH | 2-thiazolyl | 260 |
| 2-naphthyl | SO₂ | 1,2-Ph | CH═CH | 3-furanyl | 261 |
| 2-naphthyl | SO₂ | 1,2-Ph | CH═CH | 2-pyridinyl | 262 |
| 2-naphthyl | SO₂ | 1,2-Ph | CH═CH | 4-CN-Ph | 263 |
| 2-naphthyl | CH₂—O | 1,2-Ph | CH═CH | 3,5-(di-CF₃)-Ph | 264 |
| 2-naphthyl | CH₂—O | 1,2-Ph | CH═CH | 4-i-Pr-Ph | 265 |
| 2-naphthyl | CH₂—O | 1,2-Ph | CH═CH | 2,3-di-Cl-Ph | 266 |
| 2-naphthyl | CH₂—O | 1,2-Ph | CH═CH | 3,4-di-F-Ph | 267 |
| 2-naphthyl | O—CH₂ | 1,2-Ph | CH═CH | 3,5-(di-CF₃)-Ph | 268 |
| 2-naphthyl | O—CH₂ | 1,2-Ph | CH═CH | 4-i-Pr-Ph | 269 |
| 2-naphthyl | O—CH₂ | 1,2-Ph | CH═CH | 2,3-di-Cl-Ph | 270 |
| 2-naphthyl | O—CH₂ | 1,2-Ph | CH═CH | 3,4-di-F-Ph | 271 |
| 2-naphthyl | S | 1,2-Ph | CH═CH | 3,5-(di-CF₃)-Ph | 272 |
| 2-naphthyl | S | 1,2-Ph | CH═CH | 4-i-Pr-Ph | 273 |
| 2-naphthyl | S | 1,2-Ph | CH═CH | 2,3-di-Cl-Ph | 274 |
| 2-naphthyl | S | 1,2-Ph | CH═CH | 3,4-di-F-Ph | 275 |
| 2-(6-Bn—O)naphthyl | SO₂ | 1,2-Ph | CH═CH | 2-thienyl | 276 |
| 2-(6-Bn—O)naphthyl | S | 1,2-Ph | CH═CH | 2-thienyl | 277 |
| 2-(6-Bn—O)naphthyl | SO₂ | 1,2-Ph | 1,2-c-Pr | 2-thienyl | 279 |
| 2-(6-Bn—O)naphthyl | S | 1,2-Ph | 1,2-c-Pr | 2-thienyl | 279 |
| 2-(5-Bn—O)naphthyl | SO₂ | 1,2-Ph | CH═CH | 2-thienyl | 280 |
| 2-(5-Bn—O)naphthyl | S | 1,2-Ph | CH═CH | 2-thienyl | 281 |
| 2-(5-Bn—O)naphthyl | SO₂ | 1,2-Ph | 1,2-c-Pr | 2-thienyl | 282 |
| 2-(5-Bn—O)naphthyl | S | 1,2-Ph | 1,2-c-Pr | 2-thienyl | 283 |
| 2-(6-(4-CF₃)Bn—O)) | SO₂ | 1,2-Ph | CH═CH | 2-thienyl | 284 |
| naphthyl | | | | | |
| 2-(6-(4-CF₃)Bn—O)) | CH₂ | 1,2-Ph | CH═CH | 2-thienyl | 285 |
| naphthyl | | | | | |
| 2-(6-(4-CF₃)Bn—O)) | CH₂ | 1,2-Ph | 1,2-c-Ph | 2-thienyl | 286 |
| naphthyl | | | | | |
| 2-(6-(4-CF₃)Bn—O)) | CH₂ | 1,2-Ph | 1,2-c-Pr | 2-thienyl | 287 |
| naphthyl | | | | | |
| 1-(6-Bn—O)naphthyl | SO₂ | 1,2-Ph | CH═CH | 2-thienyl | 288 |
| 1-(6-Bn—O)naphthyl | CH₂ | 1,2-Ph | CH═CH | 2-thienyl | 289 |
| 2-(6-(3,4-di-F-Bn—O)) | SO₂ | 1,2-Ph | CH═CH | 2-thienyl | 290 |
| naphthyl | | | | | |
| 2-(6-(3,4-di-F-Bn—O)) | CH₂ | 1,2-Ph | CH═CH | 2-thienyl | 291 |
| naphthyl | | | | | |
| 2-(6-(4-F-Bn—O—)) | CH₂ | 1,2-Ph | 1,2-c-Pr | 2-thienyl | 292 |
| naphthyl | | | | | |
| 2-(7-Bn—O—naphthyl | SO₂ | 1,2-Ph | CH═CH | 2-thienyl | 293 |
| 2-(6-(3,4-di-F-Bn—O)) | SO₂ | 1,2-Ph | CH═CH | 3,4-di-F-Ph | 294 |
| naphthyl | | | | | |
| 2-(6-(3,4-di-F-Bn—O)) | CH₂ | 1,2-Ph | CH═CH | 3,4-di-F-Ph | 295 |
| naphthyl | | | | | |
| 2-(6-(3,4-di-F-Bn—O)) | CH₂ | 1,2-Ph | 1,2-c-Pr | 3,4-di-F-Ph | 296 |
| naphthyl | | | | | |
| 2-(7-Bn—O)naphthyl | SO₂ | 1,2-Ph | CH═CH | 3,5-(di-CF₃)-Ph | 297 |
| 2-(6-(3,4-di-F-Bn— | SO₂ | 1,2-Ph | CH═CH | 3,4-(di-CF₃)-Ph | 298 |
| O—))naphthyl | | | | | |
| 2-(6-(3,4-di-F-Bn— | CH₂ | 1,2-Ph | CH═CH | 3,4-(di-CF₃-Ph | 299 |
| O—))naphthyl | | | | | |
| 2-(7-Bn—O—naphthyl | SO₂ | 1,2-Ph | 1,2-c-Pr | 3,4-di-F-Ph | 300 |
| 2-naphthyl | CH₂—O | 1,2-Ph | CH═CH | 2-MeO-5-Br-Ph | 301 |
| 2-naphthyl | CH₂—O | 1,2-Ph-(4-Cl) | CH═CH | 2-MeO-5-Br-Ph | 302 |
| 2-naphthyl | CH₂—O | 1,2-Ph-(4-Cl) | CH═CH | 2-thienyl | 303 |
| 2-naphthyl | SO | 1,2-Ph | CH═CH | 2-MeO-5-Br-Ph | 304 |
| 2-naphthyl | SO₂ | 1,2-Ph-(4-Cl) | CH═CH | 2-MeO-5-Br-Ph | 305 |
| 2-naphthyl | O | 1,2-Ph | CH═CH | 2-MeO-5-Br-Ph | 306 |
| 2-(5-Bn—O—) | CH₂ | 1,2-Ph | CH═CH | 2-MeO-5-Br-Ph | 307 |
| naphthyl | | | | | |
| 2-(5-Bn—O—) | SO₂ | 1,2-Ph | CH═CH | 2-MeO-5-Br-Ph | 308 |
| naphthyl | | | | | |
| 2-(5-Bn—O—) | S | 1,2-Ph | CH═CH | 2-MeO-5-Br-Ph | 309 |
| naphthyl | | | | | |
| 2-naphthyl | CH₂ | 1,2-Ph | 1,2-c-Pr | 2-MeO-5-Br-Ph | 310 |
| 2-naphthyl | SO₂ | 1,2-Ph | 1,2-c-Pr | 2-MeO-5-Br-Ph | 311 |
| 2-naphthyl | S | 1,2-Ph | 1,2-c-Pr | 2-MeO-5-Br-Ph | 312 |
| 2-naphthyl | CH₂—O | 1,2-Ph | CH═CH | 2-MeO-5-Br-Ph | 313 |
| 2-naphthyl | S | 1,2-Ph | CH═CH | 2-MeO-5-Br-Ph | 314 |
| 1-(3-Me)indolyl | SO₂ | 1,2-Ph | 1,2-c-Pr | 2-MeO-5-Br-Ph | 315 |
| 1-(3-Me)indolyl | S | 1,2-Ph | 1,2-c-Pr | 2-MeO-5-Br-Ph | 316 |
| 1-(3-Me)indolyl | CH₂—O | 1,2-Ph | CH═CH | 2-MeO-5-Br-Ph | 317 |
| 1-(3-Me)indolyl | S | 1,2-Ph | CH═CH | 2-MeO-5-Br-Ph | 318 |
| 1-(3-Me)indolyl | O—CH₂ | 1,2-Ph | 1,2-c-Pr | 2-MeO-5-Br-Ph | 319 |
| 1-(3-Me)indolyl | SO | 1,2-Ph | 1,2-c-Pr | 2-MeO-5-Br-Ph | 320 |
| 1-(3-Me)indolyl | CH₂—O | 1,2-Ph-(4-Cl) | CH═CH | 2-MeO-5-Br-Ph | 321 |
| 1-(3-Me)indolyl | S | 1,2-Ph-(4-Cl) | CH═CH | 2-MeO-5-Br-Ph | 322 |
| 1-(3-Me)indolyl | SO₂ | 1,2-Ph-(4-Cl) | 1,2-c-Pr | 2-MeO-5-Br-Ph | 323 |

TABLE II

| | |
|---|---|
| | I-b |
| 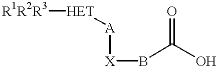 | |

| R¹R²R³-Het | A | X | B | Cpd |
|---|---|---|---|---|
| 2-naphthyl | S(O)₂ | 1,2-Ph | CH═CH | 324 |
| 2-naphthyl | S | 1,2-Ph | CH═CH | 325 |
| 4-MeS-Ph | CH₂ | 1,2-Ph | CH═CH | 326 |
| 3-Me-indolyl | CH₂ | 1,2-Ph | CH═CH | 327 |
| 3-Cl-4-F-Ph | CH₂ | 1,2-Ph | CH═CH | 328 |
| 4-Cl-Ph | CH₂ | 1,2-Ph | CH═CH | 329 |
| 2-naphthyl | CH₂ | 1,2-Ph | CH═CH | 330 |
| 2-naphthyl | S(O)₂ | 1,2-Ph | 1,2-c-Pr | 331 |
| 2-naphthyl | S(O)₂ | 1,2-Ph | CH₂—CH₂ | 332 |
| 2-naphthyl | S | 1,2-Ph | CH═CH | 333 |
| 3,4-di-Cl-Ph | S(O)₂ | 1,2-Ph | CH₂—CH₂ | 334 |
| 3,4-di-Cl-Ph | CH₂ | 1,2-Ph | CH═CH | 335 |
| 2-(6-Bn—O—)naphthyl | CH₂ | 1,2-Ph | CH═CH | 336 |
| 2-(6-Bn—O—)naphthyl | CH₂ | 1,2-Ph | 1,2-c-Pr | 337 |
| 2-(6-Bn—O—)naphthyl | SO₂ | 1,2-Ph | 1,2-c-Pr | 338 |
| 2-(6-Bn—O—)naphthyl | CH₂—O | 1,2-Pr | 1,2-c-Pr | 339 |
| 2-(6-Bn—O—)naphthyl | O—CH₂ | 1,2-Ph | 1,2-c-Pr | 340 |
| 2-(6-Bn—O—)naphthyl | SO₂ | 1,2-Ph | CH═CH | 341 |
| 2-(6-Bn—O—)naphthyl | CH₂—O | 1,2-Ph | CH═CH | 342 |
| 2-(6-Bn—O—)naphthyl | O—CH₂ | 1,2-Ph | CH═CH | 343 |
| 2-(6-Bn—O—)naphthyl | S | 1,2-Ph | CH═CH | 344 |
| 2-(6-Bn—O—)naphthyl | SO₂ | 1,2-Ph | CH═CH | 345 |
| 2-(6-(4-CF₃) | CH₂ | 1,2-Ph | CH═CH | 346 |
| Bn—O—)naphthyl | | | | |

C = —S(CH₂)₂Ph, D = —O(CH₂)₃—O, Qn = 2-(7-chloroquinolin-2-yl)ethenyl Bn = benzyl

Compounds that serve as E-type prostaglandin ligands are also found in U.S. application Ser. No. 60/103,371 (Merck Case No. 20085PV) filed on Oct. 7, 1998, and addressing compounds of structural Formula IV below:

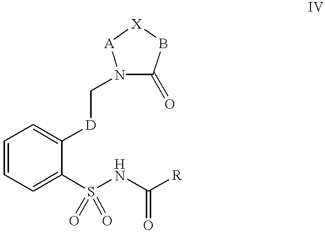

wherein

A and B are independently unsubstituted, monosubstituted or disubstituted ortho-benzenediyl or ortho-heteroarylenediyl wherein the substituents are selected from the group consisting of:

halogen,

C₁₋₅ alkyl,

C₁₋₅ alkoxy,

C₁₋₅ alkylthio,

nitro,

CN,

C₁₋₅ fluoroalkyl,

COOR³, and

NR³₂;

X is CH₂CH₂, CH═CH, CH₂Y, YCH₂, CH₂CH₂CH₂, ortho-benzenediyl or ortho-heteroarylenediyl;

Y is O, S, CF₂, or C═O;

D is unsubstituted, monosubstituted, or disubstituted benzendiyl wherein the substituents are selected from:

halogen,

C₁₋₅ alkyl,

C₁₋₅ alkoxy,

C₁₋₅ alkylthio,

nitro,

CN,

C₁₋₅ fluoroalkyl,

COOR³, and

NR³₂;

R is:

C₁₋₆ alkyl,

(CR¹R²)ₙO—Ph,

(CR¹R²)ₙO-heteroaryl,

O—(CR¹R²)ₙPh,

O—(CR¹R²)nheteroaryl,

NR³—(CR¹R²)ₙPh,

NR³—(CR¹R²)nheteroaryl,

C₂₋₆ alkenyl-Ph,

C₂₋₆ alkenyl-heteroaryl,

(CR¹R²)ₙPh, or

(CR¹R²)nheteroaryl,

wherein Ph or heteroaryl is unsubstituted, monosubstituted or disubstituted with substituents selected from:

halogen,

C₁₋₅ alkyl,

C₁₋₅ alkoxy,

C₁₋₅ alkylthio,

nitro,

CN,

C₁₋₅ fluoroalkyl,

COOR³, and

NR³2;

n=0, 1, 2 or 3;

R¹ and R² are independently hydrogen, C₁₋₃ alkyl, benzyl, C1-3 fluoroalkyl, C₁₋₃ alkoxy, or fluorine;

R³ is H or C₁₋₆ alkyl.

METHODS OF SYNTHESIS

The compounds described above can be prepared according to the following methods. Other synthetic routes will be immediately apparent to those skilled in the art.

Preparation of intermediates:

Biphenyl sulfonamides:

As shown in Scheme I, 2-bromobenzenesulfonyl chloride III (purchased from Lancaster) is reacted withtert-butylamine. The resulting sulfonamide IV is converted, via a palladium-catalyzed coupling with boronic acid V (purchased from Omega Chemical Company Inc.) to biphenyl derivative VI. When treated with HBr in acetic acid, activation of the hydroxyl group and deprotection occur in the same procedure to afford sulfonamide VII. This sulfonamide is a common intermediate used in alkylation reactions with azocinones (dibenzolactams).

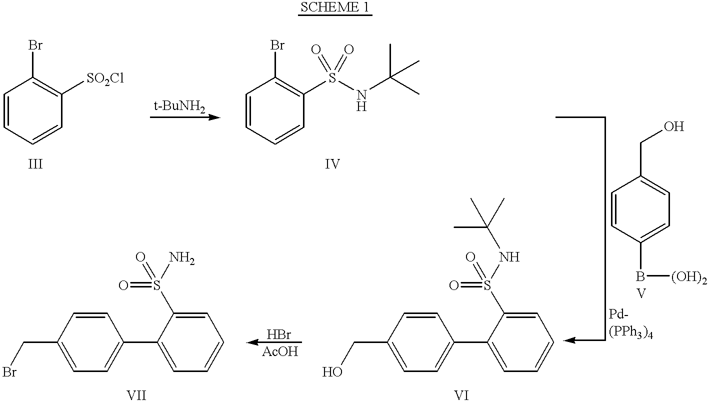

Substituted boronic acids can also be prepared

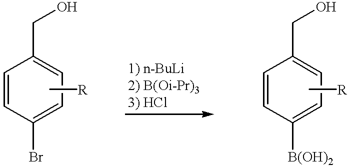

Synthesis of comnounds

Azocinones (dibenzolactams):

Tetrahydrodibenz[b,f]azocin-6-one (VIII), shown in Scheme 2, is commercially available from Aldrich Chemical Co., Inc., in Milwaukee, Wis. The corresponding unsaturated compound IX can be prepared (VIII can also be prepared in the same manner from dibenzosuberone) from commercially available dibenzosuberenone (X) via a two-step sequence (i- oxime formation using hydroxylamine and ii- Beckmann rearrangement on the corresponding tosylate) as shown in Scheme 2.

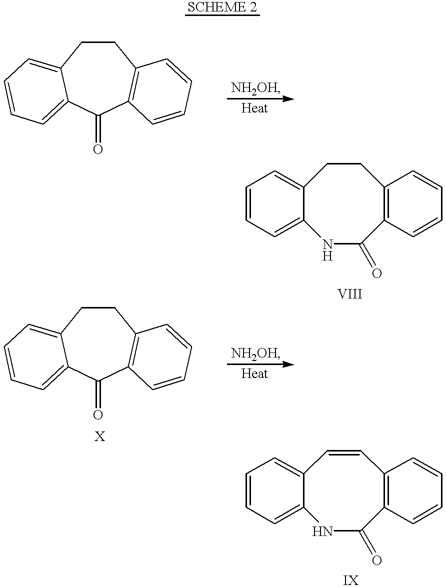

As depicted in Scheme 3, other dibenzolactam and heteroarylenediyl derivatives can be prepared via a three-step sequence: (I) palladium-catalyzed Heck reaction; (ii) hydrogenation and (iii) cyclization induced by 1-hydroxybenzotriazole hydrate (HOBT), 1-(3-dimethylaminopropyl)-3-ethylcarbodiimide hydrochloride (EDCl) and potassium hydride. For example, fluorinated derivatives XI and XII are prepared from the reaction of styryl derivative XV and anilines XIII and XIV, respectively (both purchased from Lancaster). Heteroaryl starting materials related to XV can also be prepared using the Heck reaction on the corresponding heteroaryl bromide and ethylene.

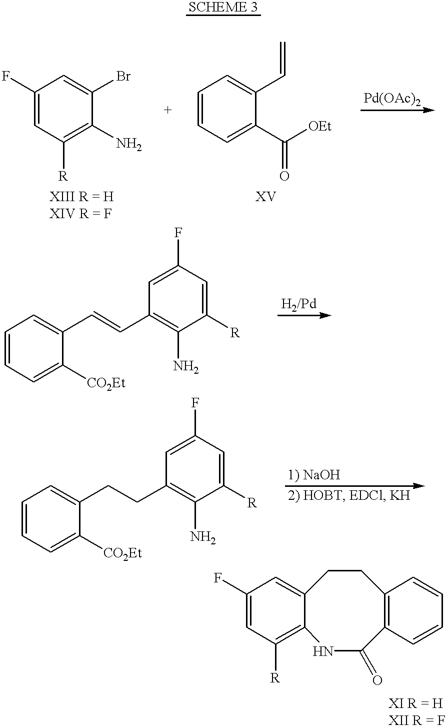

Alternatively, compound VIII can be converted to VIIIA and subsequently to VIIIB via a benzylic bromination reaction using N-bromosuccinimide (NBS) outlined in Scheme 4 and described in J. Org. Chem. 1972, p. 4907. This intermediate can in turn be transformed to VIIIC using standard procedures and VIIIE can be obtained from VIIIC following one of many protocol for carbonyl transposition (PhCHO,OH—/LiAlH₄, AICl₃/O₃). These isomers can then each be transformed to the difluoro analog VIIID and VIIIF by reaction with DAST (diethylaminosulfur trifluoride). The lactams corresponding to products VIIID and VIIIF can then be obtained using standard hydrolytic procedures. Other lactams described herein can be prepared according to published procedures and/or are commercially available.

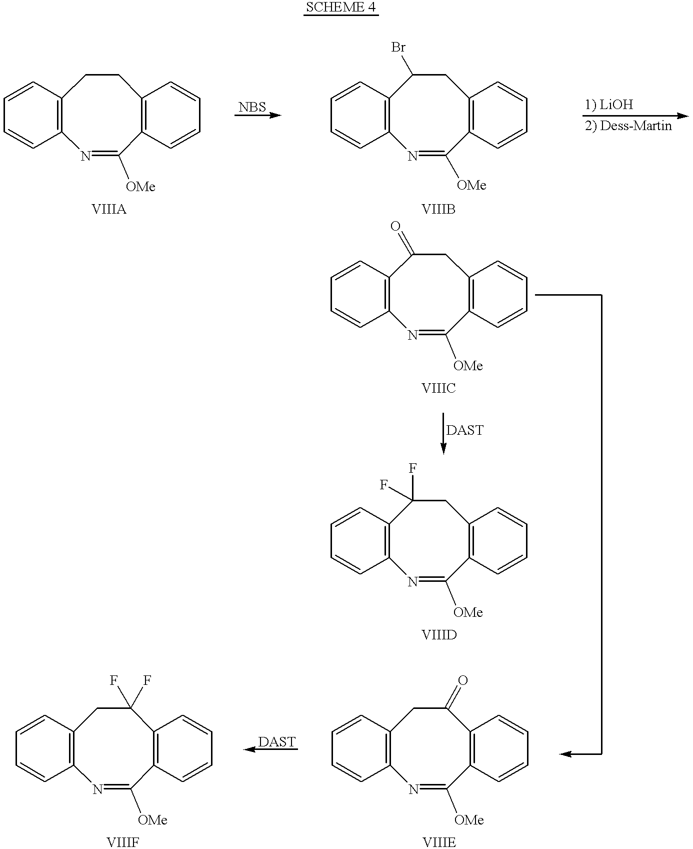

As shown in Scheme 5, dibenzolactam VIII was then treated with sodium hydride and sulfonamide VII to provide biphenyl derivative XVI which serves as a common intermediate for the synthesis of several of the compounds of the present invention. Alternatively, dibenzolactam VIII can be replaced by any of the lactams IX, XI or XII and reacted with VII. Compound XVI can then be transformed to several compounds depending on the choice of the acid chlorides used. For example, treatment of XVI with hydrocinnamoyl chloride and Hunïg's base in DMF (dimethylformamide) provides acid sulfonamide XVII.

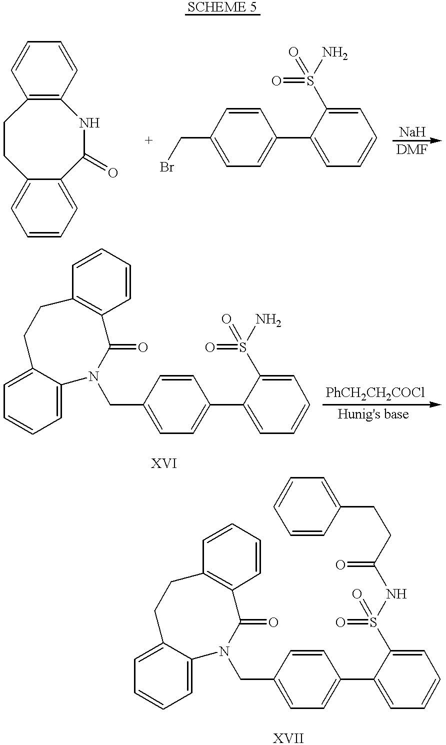

Preparation of intermediates 2-bromo tert-butylbenzenesulfonamide IV

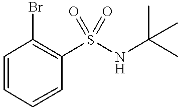

Tert-butylamine (30 mL, 0.29 mol) was slowly added to a solution of 2-bromobenzenesulfonyl chloride (30 g, 0.11 mol) with mechanical stirring at room temperature. After four hours, the precipitate was filtered and the solvent was evaporated to afford the sulfonamide. ¹H nmr (400 MHz, CDCl₃) δppm 8.15 (1H, dd, J=10.5, 2.0 Hz), 7.68 (1H, dd, J=10.5, 2.0 Hz), 7.40 (1H, m), 7.31 (1H, m), 5.15 (1H, br. s), 1.18 (9H, s).

Hydroxymethyl biphenysulfonamide VI

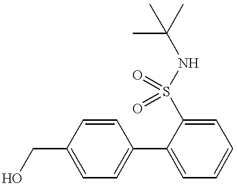

A degassed solution of 2-bromo tert-butylbenzenesulfonamide (IV) (15.6 g, 53.5 mmol) and tetrakis(triphenylphosphine)palladium (3.1 g, 2.7 mmol) in dimethoxyethane (270 mL) was stirred at room temperature for 5 minutes. Boronic acid V (purchased from Omega Chemical Company Inc.) (10 g, 53.5 mmol) and a 2M solution of sodium bicarbonate (53 mL) were then added and the mixture was heated to 90° C. and stirred at this temperature for 24 hours. The mixture was then cooled down and a saturated solution of ammonium chloride (300 mL) and ethyl acetate (300 mL) were added. The separated aqueous layer was extracted with ethyl acetate (3 x 100 mL) and the combined organic layers were dried (MgSO₄ anh.), filtered and evaporated. Flash chromatography of the residue (EtOAc-hexanes 1:1) yielded biphenyl compound VI. ¹H nmr (400 MHz, CDCl₃) δppm 8.15 (1H, dd, J=10.5, 2.0 Hz), 7.50 (6H, m), 7.30 (1H, m), 4.72 (2H, m), 3.61 (1H, m), 1.91 (1H, m), 1.00 (9H, s).

Bromomethyl biphenyl derivative VII

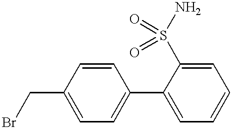

Compound VII can be prepared according to the following two alternative methods:

Method 1

A solution of hydrobromic acid (48%, 75 mL) was added to a solution of alcohol VI (22.3 g, 69.8 mmol) in acetic acid (75 mL) at room temperature. The mixture was heated to 110 ° C. and stirred at this temperature for 2.5 hours. After cooling to room temperature, ethanol (100 mL) and toluene (100 mL) were added and the resulting mixture was evaporated under reduced pressure. The residue was dissolved in ethyl acetate and neutralized with saturated aqueous NaHCO₃. The separated aqueous layer was washed with brine, dried (MgSO₄ ), filtered and evaporated.

Alternatively, compound VII was prepared according to the following two-step procedure.

Method 2

At 0° C., carbon tetrabromide (12.5 g, 37.6 mmol) was added to compound VI (10 g, 31.3 mmol) in dichloromethane (100 mL). Bis(diphenylphosphino)ethane (7.5 g, 0.6 mmol) was then added portionwise. The mixture was stirred at 0° C. for 12 hours and it was then poured into dry ether (750 mL), filtered over Celite and evaporated. Trifluoroacetic acid (100 mL) was then added and the resulting mixture was evaporated under reduced pressure. The residue was recrystallized from hexanes.

Representative examples of compounds which can be made in accordance with the above procedures are set forth below.

| | |
|---|---|
| | IV |
| 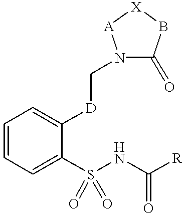 | |

| A | B | D | X | R |
|---|---|---|---|---|
| 1,2-Ph | 1,2-Ph | 1,4-Ph | CH₂CH₂ | CH₂CH₂Ph |
| 1,2-Ph | 1,2-Ph | 1,4-Ph | CH₂CH₂ | NH(CH₂)₂Ph |
| 1,2-Ph | 1,2-Ph | 1,4-Ph | CH₂CH₂ | NH(CH₂)₃Ph |
| 1,2-Ph | 1,2-Ph | 1,4-Ph | CH₂CH₂ | OCH₂Ph |
| 1,2-Ph | 1,2-Ph | 1,4-Ph | CH₂CH₂ | CH₂OPh |
| 1,2-Ph | 1,2-Ph | 1,4-Ph | CH₂CH₂ | CH₂Ph |
| 1,2-Ph | 1,2-Ph | 1,4-Ph | CH₂CH₂ | C(CH₃)₂Ph |
| 1,2-Ph | 1,2-Ph | 1,4-Ph | CH₂CH₂ | CH(CH₃)Ph |
| 1,2-Ph | 1,2-Ph | 1,4-Ph | CH₂CH₂ | C(CH₃)₂CH₂Ph |
| 1,2-Ph | 1,2-Ph | 1,4-Ph | CH═CH | C(CH₃)₂CH₂Ph |
| 4-F,1,2-Ph | 1,2-Ph | 1,4-Ph | CH₂CH₂ | C(CH₃)₂CH₂Ph |
| 4,6-F,1,2- | 1,2-Ph | 1,4-Ph | CH₂CH₂ | C(CH₃)₂CH₂Ph |
| Ph | | | | |
| 1,2-Ph | 1,2-Ph | 1,4-Ph | CH₂CH₂ | (S)-C(CF₃)(OCH₃)Ph |
| 1,2-Ph | 1,2-Ph | 1,4-Ph | CH₂CH₂ | (R)- |
| | | | | C(CF₃)(OCH₃)Ph |
| 1,2-Ph | 1,2-Ph | 1,4-Ph | CH₂CH₂ | NCH₃(CH₂)₂Ph |
| 1,2-Ph | 1,2-Ph | 1,4-Ph | CH₂CH₂ | (S)-NHCH(CH₃)Ph |
| 1,2-Ph | 1,2-Ph | 1,4-Ph | CH₂CH₂ | NH(CH₂)₂2- |
| | | | | Thiophene |
| 1,2-Ph | 1,2-Ph | 1,4-Ph | CH₂CH₂ | (E)-CH═CHPh |
| 1,2-Ph | 1,2-Ph | 1,4-Ph | CH₂CH₂ | NH(CH₂)₃CH₃ |
| 5-Cl,1,2-Ph | 1,2-Ph | 1,4-Ph | OCH₂ | NH(CH₂)₂2- |
| | | | | Thiophene |
| 1,2-Ph | 1,2-Ph | 1,4-Ph | CH₂CH₂ | NHC(CH₃)₃ |
| 1,2-Ph | 1,2-Ph | 1,4-Ph | CH₂CH₂ | NHCH₂Ph |
| 1,2-Ph | 1,2-Ph | 1,4-Ph | CH₂CH₂ | o-Cl-Ph |
| 1,2-Ph | 1,2-Ph | 1,4-Ph | OCH₂ | NH(CH₂)₂2- |
| | | | | Thiophene |

Examples of COX-2 selective inhibitors are found in the following patents and published applications: WO96/25405, U.S. Pat. No. 5,633,272, WO 97/38986, U.S. Pat. No. 5,466,823, WO 98/03484, WO 97/14691 and WO 95/00501.

Some of the compounds used in the present invention contain one or more asymmetric centers and may thus give rise to diastereomers and optical isomers. The present invention is meant to include all such possible diastereomers as well as their racemic and resolved, enantiomerically pure forms and pharmaceutically acceptable salts thereof.

Some of the compounds used in the present invention contain olefinic double bonds, and unless specified otherwise, are meant to include both E and Z geometric isomers.

The compounds useful herein also include pharmaceutically acceptable salts. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic, manganous, potassium, sodium, zinc salts, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When a compound used in the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It is understood that in the methods of treatment which follow, references to the compounds are meant to also include pharmaceutically acceptable salts and hydrates.

Dose Ranges

The magnitude of a prophylactic or therapeutic dose of the E-type prostaglandin varies with the nature and the severity of the condition to be treated, the particular compound and its route of administration. It also varies according to factors including the age, weight, general health, sex, diet, time of administration, rate of excretion, drug combination and response of the individual patient. In general, a daily dose of from about 0.001 mg to about 100 mg per kg body weight of a mammal, preferably 0.01 mg to about 10 mg per kg is useful. On the other hand, it may be necessary to use dosages outside these limits in some cases.

The COX-2 selective inhibitor used will similarly vary in dosage, depending upon the nature and the severity of the condition to be treated and with the particular compound and its route of administration. Generally daily dosage ranging from as low as about 0.01 mg to about 140 mg/kg of body weight per day are useful in the treatment of the indicated conditions, or alternatively about 0.5 mg to about 7 g per patient per day. For example, inflammation may be effectively treated by the administration of from about 0.01 to 50 mg of the compound per kilogram of body weight per day, or alternatively about 0.5 mg to about 3.5 g per patient per day.

Pharmaceutical Compositions

In the pharmaceutical composition described herein, the active ingredients can be combined with the carrier materials to produce a single dosage form. For example, a formulation intended for oral administration to humans may contain from as low as about 0.5 mg to as high as about 5 g of the active agents, compounded with an appropriate and convenient amount of carrier material which may vary from about 5 to about 95 percent of the total composition. Dosage units will generally contain between from about 1 mg to about 2 g of the active ingredients, typically about 25 mg, 50 mg, 100 mg, 200 mg, 300 mg, 400 mg, 500 mg, 600 mg, 800 mg or 1000 mg of the actives.

For the treatment or prevention of any of the prostanoid and/or COX-2 mediated diseases, the compounds may be administered separately or together, orally, topically, parenterally, by inhalation spray or rectally in dosage unit formulations containing conventional non-toxic pharmaceutically acceptable carriers, adjuvants and vehicles.

The term parenteral as used herein includes subcutaneous injections, intravenous, intramuscular, intrasternal injection or infusion techniques. In addition to the treatment of warm-blooded animals such as mice, rats, horses, cattle, sheep, dogs, cats and the like, the combination of compounds of the invention is useful in the treatment of humans.

The pharmaceutical compositions containing the active ingredients may be in a form suitable for oral use, for example, as tablets, troches, lozenges, aqueous or oily suspensions, dispersible powders or granules, emulsions, hard or soft capsules, syrups or elixirs. Compositions intended for oral use may be prepared according to any method known to the art for the manufacture of pharmaceutical compositions and such compositions may contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water-miscible solvents such as propylene glycol, PEGs and ethanol, or an oil medium, for example peanut oil, liquid paraffin, or olive oil.

Aqueous suspensions containing the active compounds in admixture with excipients suitable for the manufacture of aqueous suspensions. Such excipients are suspending agents, for example sodium carboxymethylcellulose, methylcellulose, hydroxypropyl methylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. Aqueous suspensions typically contain one or more preservatives, for example ethyl, or n-propyl, p-hydroxybenzoate, one or more colouring agents, one or more flavouring agents, and/or one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Examples of suitable dispersing or wetting agents and suspending agents are mentioned above. Additional excipients, for example sweetening, flavouring and colouring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents.

The pharmaceutical compositions may be in the form of a sterile injectable aqueous or oleagenous suspension. This suspension may be formulated using those suitable dispersing or wetting agents and suspending agents which have been mentioned above. The preparation may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. Cosolvents such as ethanol, propylene glycol or polyethylene glycols may also be used. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The composition may also be in the form of suppositories for rectal administration. These compositions can be prepared by mixing the drug with a suitable non-irritating excipient which is solid at ambient temperatures but liquid at the rectal temperature and will therefore melt in the rectum to release the drugs. Examples of such materials are cocoa butter and polyethylene glycols.

For topical use, creams, ointments, gels, solutions or suspensions, etc., containing the compounds are employed. Topical applications include mouth washes and gargles. Topical formulations are generally comprised of a pharmaceutical carrier that includes cosolvents, emulsifiers, penetration enhancers, preservatives and emollients.

The composition of the present invention may also include additional therapeutic agents. For example, conventional analgesics such as aspirin or acetaminophen may be incorporated into the composition. Other examples of additional therapeutic agents which can be included are NSAIDs, such as ibuprofen or naproxen, and other compounds.

Utilities

The ability of the E-type prostaglandin ligand to interact with prostaglandin receptors makes them useful for preventing or reversing undesirable symptoms caused by prostaglandins in a mammalian, especially human, subject. This mimicking or antagonism of the actions of prostaglandins indicates that the compounds and pharmaceutical compositions are useful to treat, prevent, or ameliorate in mammals and especially in humans pain, fever and inflammation of a variety of conditions including rheumatic fever, symptoms associated with influenza or other viral infections, common cold, low back and neck pain, skeletal pain, post-partum pain, dysmenorrhea, headache, migraine, toothache, sprains and strains, myositis, neuralgia, synovitis, arthritis, including rheumatoid arthritis, degenerative joint diseases (osteoarthritis), gout and ankylosing spondylitis, bursitis, burns including radiation and corrosive chemical injuries, sunburns, pain following surgical and dental procedures as well as immune and autoimmune diseases.

In addition, such a compound may inhibit cellular neoplastic transformations and metastatic tumor growth and hence can be used in the treatment of cancer. Such compounds are also of use in the treatment and/or prevention of prostaglandin-mediated proliferation disorders such as diabetic retinopathy and tumor angiogenesis.

The E-type prostaglandin ligands inhibit prostanoid-induced smooth muscle contraction by antagonizing contractile prostanoids or mimicking relaxing prostanoids and henceare of use in the treatment of dysmenorrhea, premature labor, asthma and eosinophil related disorders. The compounds are also of use in the treatment of Alzheimer's disease, the treatment of glaucoma, for the prevention of bone loss (treatment of osteoporosis) and for the promotion of bone formation (treatment of fractures) and other bone diseases such as Paget's disease.

Similarly, the COX-2 selective inhibitors are useful in a wide array of diseases and conditions, including without limitation:

relief of pain, fever and inflammation due to a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, burns, injuries, following surgical and dental procedures.

inhibiting cellular neoplastic transformations and metastic tumor growth and hence can be used in the treatment of cancer. inhibiting cyclooxygenase-mediated proliferative disorders such as diabetic retinopathy and tumour angiogenesis.

inhibiting prostanoid-induced smooth muscle contraction by preventing the synthesis of contractile prostanoids and hence may be of use in the treatment of dysmenorrhea, premature labor, asthma and eosinophil related disorders.

treating or preventing Alzheimer's disease,

treating or preventing bone loss (treatment of osteoporosis) and treating or preventing glaucoma.

A preferred method of treatment or prevention described herein for the combination of an E-type prostaglandin ligand and a COX-2 selective inhibiting compound is for the treatment, prevention or relief of pain, fever and inflammation.

Another preferred utility for the combination of an E-type prostaglandin ligand and a COX-2 selective inhibiting compound is for the treatment of dysmenorrhea, premature labor, asthma and eosinophil related disorders.

The combination is particularly useful as an alternative to conventional non-steroidal antiinflammatory drugs, particularly where such non-steroidal antiinflammatory drugs are contraindicated, such as in patients with peptic ulcers, gastritis, regional enteritis, ulcerative colitis, diverticulitis or with a recurrent history of gastrointestinal lesions; GI bleeding, coagulation disorders including anemia such as hypoprothrombinemia, haemophilia or other bleeding problems; kidney disease; those prior to surgery or taking anticoagulants.

Similarly, the combination is useful as a partial or complete substitute for conventional NSAIDs in preparations wherein they are presently co-administered with other agents or ingredients. Thus, the invention encompasses pharmaceutical compositions and methods for treating E-type prostaglandin or COX-2 mediated diseases as defined above, further comprising administering one or more ingredients such as another pain reliever including acetominophen or phenacetin; a potentiator including caffeine; an H₂-antagonist, aluminum or magnesium hydroxide, simethicone, a decongestant including phenylephrine, phenylpropanolamine, pseudophedrine, oxymetazoline, ephinephrine, naphazoline, xylometazoline, propylhexedrine, or levo-desoxyephedrine; an antiitussive including codeine, hydrocodone, caramiphen, carbetapentane, or dextramethorphan; a prostaglandin including misoprostol, enprostil, rioprostil, ornoprostol or rosaprostol; a diuretic; a sedating or non-sedating antihistamine. In addition the invention encompasses a method of treating cyclooxygenase mediated diseases comprising: administration to a patient in need of such treatment an effective amount of the E-type prostaglandin ligand and a COX-2 selective inhibiting compound, optionally coadministered with one or more of such ingredients as listed immediately above.

More particularly, a method of treating or preventing an E-type prostaglandin or COX-2 mediated disease or condition is addressed wherein the disease is selected from the group consisting of:

pain, fever, inflammation, rheumatic fever, symptoms associated with influenza or other viral infections, common cold, low back and neck pain, skeletal pain, post-partum pain, dysmenorrhea, headache, migraine, toothache, sprains, strains, myositis, neuralgia, synovitis, arthritis including rheumatoid arthritis, degenerative joint diseases (osteoarthritis), gout, ankylosing spondylitis, bursitis, burns including radiation and corrosive chemical injuries, sunburns, pain following surgical and dental procedures, immune and autoimmune diseases, cellular neoplastic transformations, metastatic tumor growth, prostaglandin-mediated proliferation disorders such as diabetic retinopathy and tumor angiogenesis, dysmenorrhea, premature labor, asthma, eosinophil related disorders, Alzheimer's disease, glaucoma, bone loss (osteoporosis), promotion of bone formation (treatment of fractures) and other bone diseases such as Paget's disease.

The compounds useful herein can be synthesized as described in the above mentioned patents and patent applications.

Utility for the compounds is described in connection with the following test procedures.

Methods—Carrageenan-induced paw hvperalaesia in rats

Male Sprague Dawley rats (90-110 g) were fasted overnight before use. At approximately 10:00 am, the rats were injected intraplantarly in a hind paw with 150 4l 3% carrageenan (4.5 mg carrageenan/paw). A group of control rats was injected with an equivalent volume of saline (150 4l per paw). Two hours later, the saline-injected rats were dosed orally with a vehicle (0.5% methocel). The carrageenan-injected rats were dosed orally with either a vehicle (0.5% methocel) or a test compound. The following treatment groups were included in each experiment: COX-2 inhibitor alone at 0.3, 1, 3 and 10 mg/kg; EP₃ antagonist alone at a fixed dose (5 mg/kg); a fixed dose of EP₃ antagonist (5 mg/kg) in combination with a COX-2 inhibitor at 0.3, 1, 3 or 10 mg/kg. In another dosing regimen, the dose of the COX-2 inhibitor was fixed. In such case, the following treatment groups were included: EP₃ antagonist alone at 0.3, 1, 3 and 10 mg/kg; COX-2 inhibitor alone at a fixed dose; a fixed dose of COX-2 inhibitor in combination with a EP3 antagonist at 0.3, 1, 3 or 10 mg/kg. Responses to mechanical stimuli were measured before injection of carrageenan (baseline value at time zero), and again at 1 hour after oral administration of the test compound (i.e., 3 hr after injection of carrageenan) using an analgesia meter (Ugo Basile). Vocalization or struggle behaviour was used as an indication for nociceptive response. Percent hyperalgesia was calculated using the value in the saline-injected group as 0% hyperalgesia and that in the carrageenan-injected vehicle-treated group as 100 % hyperalgesia.

The following compounds were used:

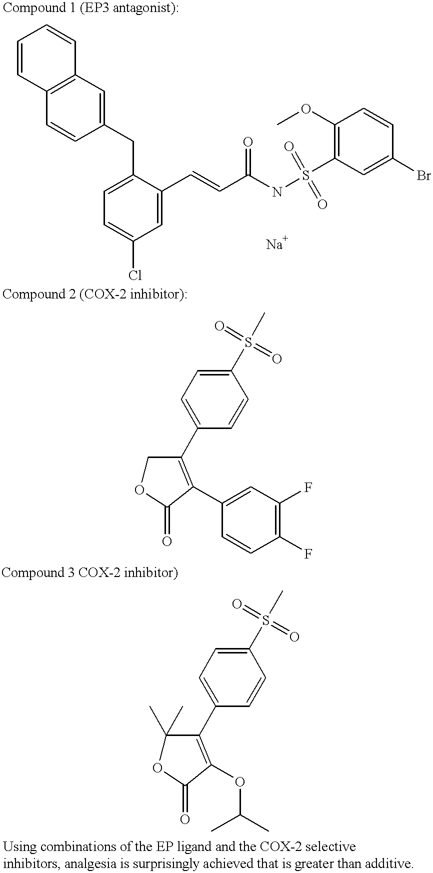

The compositions and methods described herein also in particular include antiinflammatory compositions and a method of treating inflammation using the combinations described. The method of treating inflammation can be demonstrated using the following general procedure.

Methods—Carrageenan-induced paw edema in rats

Male Sprague-Dawley rats (180-200g) were fasted overnight prior to oral administration of 1 ml of either the vehicle (0.5% methocel) or a test compound. The following treatment groups were included: COX-2 inhibitor (compound 2) alone at 0.1, 0.3, 1, 3, 10 or 30 mg/kg; EP₃ antagonist (compound 1) alone at a fixed dose (3 mg/kg); a fixed dose of EP₃ antagonist (3 mg/kg, compound 1) in combination with a COX-2 inhibitor (compound 2) at 0.1, 0.3, 1, 3, 10 or 30 mg/kg. One hr later, a line was drawn using a permanent marker at a level above the ankle in one hind paw to define the area of the paw to be monitored. The paw volume (Vo) was measured using a plethysmometer (Ugo- Basile). The animals were then injected subplantarly with 0.1 ml of a 1% carrageenan solution in saline (i.e. 1 mg carrageenan per paw). Three hr later, the paw volume (V3) was measured and the increases in paw volume (V₃-V₀) were calculated. Paw edema in the treated group was compared to that observed in the vehicle-control group. Percent inhibition was calculated taking the values in the control group as 0 %. All treatment groups were coded to eliminate bias from the observer.

Using the above procedure, it is demonstrated that the combinations of compounds are effective in treating inflammation, and that using the combination of an E-type prostaglandin ligand and a COX-2 selective inhibiting compound, the effect is greater than additiive.

What is claimed is:

1. A pharmaceutical composition which is comprised of an E-type prostaglandin ligand and a COX-2 selective inhibiting compound, in combination with a pharmaceutically acceptable carrier.

2. A pharmaceutical composition in accordance with claim 1 wherein each of the E-type prostaglandin and COX-2 selective inhibiting compounds is present in an amount ranging from about 1 mg to about 2 g of each of the compounds.

3. A method of treating or preventing a prostaglandin and COX-2 mediated disease or condition in a mammalian patient, comprising administering to said patient an amount of a E-type prostaglandin ligand and a COX-2 selective inhibiting compound in an amount which is effective to treat or prevent said disease or condition.

4. A method of treating or preventing pain in a mammalian patient, comprising administering to said patient an amount of a E-type prostaglandin ligand and a COX-2 selective inhibiting compound in an amount which is effective to treat or prevent pain.

5. A method of treating or preventing inflammation in a mammalian patient in need thereof, comprising administering to said patient an amount of a E-type prostaglandin ligand and a COX-2 selective inhibiting compound which is effective to treat or prevent inflammation.
